(19) United States
(12) Patent Application Publication
(10) Pub. No.: US 2021/0327538 A1
(43) Pub. Date: Oct. 21, 2021

(54) METHODS AND SYSTEMS FOR CALLING PLOIDY STATES USING A NEURAL NETWORK

(71) Applicant: Natera, Inc., San Carlos, CA (US)

(72) Inventors: Agust EGILSSON, Kopavogur (IS); George GEMELOS, Portland, OR (US); Styrmir SIGURJONSSON, San Jose, CA (US)

(73) Assignee: Natera, Inc., San Carlos, CA (US)

(21) Appl. No.: 17/252,205

(22) PCT Filed: Jul. 16, 2019
(86) PCT No.: PCT/US2019/041981

§ 371 Date: Dec. 14, 2020

Related U.S. Application Data
(60) Provisional application No. 62/699,135, filed on Jul. 17, 2018.

(51) Int. Cl. G16B 20/20; C12Q 1/6874; G16B 30/00; C12Q 1/6883

(52) U.S. Cl. CPC G16B 20/20; C12Q 1/6874; C12Q 2600/156; C12Q 1/6883; G16B 30/00

(57) ABSTRACT

A method of calling a ploidy state using a neural network includes determining, for a training sample, genetic sequencing data or genetic array data for a plurality of genetic positions, determining respective true ploidy state values for a plurality of genetic segments, each genetic segment respectively comprising at least some of the plurality of genetic positions, based on the genetic sequencing data or genetic array data, and determining a neural network comprising one or more layers for calling respective ploidy state values, the neural network defined at least in part by a plurality of weights. The method further includes iteratively modifying the weights using specific processes. The method further includes calling, for a test sample, a ploidy state for a target genetic region by propagating genetic sequencing data for the test sample or genetic array data for the test sample through the modified neural network.

### CROSS REFERENCE To RELATED APPLICATIONS

This application claims priority to U.S. Provisional Application No. 62/699,135 filed Jul. 17, 2018, which is hereby incorporated by reference in its entirety.

### BACKGROUND OF THE DISCLOSURE

Detecting embryonic chromosomal abnormalities can be helpful in determining the health of an embryo or fetus. For example, the health of the embryo can be determined prior to implantation via an In Vitro Fertilization (IVF) process by detecting aneuploidies, including whole chromosome aneuploidies or regional aneuploidies, or the health of a fetus in terms of aneuploidies can be determined using non-invasive prenatal testing (NIPT). However, it can be difficult to detect such aneuploidies using conventional techniques, and it can be difficult to detect such aneuploidies with granularity with regard to locations of the aneuploidies. The present disclosure describes improved systems and methods that provide for, among other things, accurately calling embryonic and fetal aneuploidies, and calling embryonic and fetal aneuploidies for a particular segment of a chromosome.

### SUMMARY OF THE DISCLOSURE

At least some of the systems and methods described herein relate to calling embryonic or fetal aneuploidies using a neural network. The neural network can be trained on annotated data to accurately call a ploidy state of an embryonic sample, thus providing insight into the health of the embryo. The systems and methods herein can provide for improved detection, location and classification of aneuploidies in embryos and fetuses, both from array and sequencing data, including aneuploidies that are specific to small segments of a chromosome, and can provide for classification of each genomic position by ploidy state in addition to classifying larger ploidy regions. The systems and methods described herein may implement deep learning or machine learning processes, such as any of those described in the publication Deep Learning (Adaptive Computation and Machine Learning), Ian Goodfellow, Yoshua Bengio, Aaron Courville, MIT Press (Nov. 18, 2016), which is incorporated herein in its entirety.

The systems and methods described herein can provided for improved non-invasive prenatal testing can be used to test for many conditions; to determine whether or not a fetus has any whole chromosomal abnormalities such as Down syndrome, Edwards syndrome, or Turner Syndrome, to determine whether or not a fetus has any partial chromosomal abnormalities such as mosaicism, deletion syndromes, or duplications, or to determine the genotype of the fetus at one or a plurality of loci, for example disease linked single nucleotide polymorphisms (SNPs). Furthermore, the systems and methods described herein can provided for improved pre-implantation genetic diagnosis (PGD). PGD can detect chromosomal abnormalities such as aneuploidy, and can be used to ensure successful implantation and a healthy baby. PGD can also be sued for genetic disease screening.

Some embodiments described herein are directed to systems and methods for calling and simulating the ploidy state of a chromosome segment by training and employing neural networks. The chromosomal segments being called are represented by targeted sequencing or array data obtained from plasma mixtures and genomic samples. The neural network training methods describe herein are directed to whole chromosome aneuploidy calling and to calling aneuploidies present on sub-chromosomal level. The methods improve existing algorithms, allow the neural networks to learn genomic location biases and add robustness and invariance to noise by altering the training pipelines. A system for simulating realistic segmental ploidy states by first capturing the presence of common homologs in the population is taught and employed to augment the training data enabling the trained neural network to call deletions, such as small microdeletions, in the chromosomal structures. A test sample can be passed through the neural network to determine characteristics of the test sample, including detection of genetic abnormalities.

In some implementations, the neural network takes as inputs genetic data for maternal and paternal genetic data in addition to the embryonic genetic data. The genetic data may be, for example, reads or sequencing of strands or fragments of DNA or RNA of any type, or data derived therefrom. The neural network can be developed using training data that includes embryonic, maternal and paternal genetic data, and by making use of such data can accurately call a ploidy state of the embryonic sample. As used herein, the term “ploidy state” can refer to a categorization of a genetic segment or chromosome being euploid, or aneuploid, and can refer to a genetic segment or chromosome exhibiting a particular aneuploidy. In some implementations, the neural network is trained using augmented data that includes one or more synthetic cases. For example, the augmented data may include genetic information generated by combining two other genetic segments included in the training data, or may include genetic information generated by simulating a deletion in a genetic segment included in the training data. The synthetic cases may be specifically generated to include an aneuploidy, and a set of “true” or known values (e.g. determined by manual annotation) may be updated to account for the synthetic cases. Use of the synthetic cases in training can provide for a neural network readily able to call a sub-chromosomal aneuploidy, far more efficiently and accurately than some other techniques.

Accordingly, in one aspect, the present disclosure provides a method of conducting prenatal testing, including determining, for a training sample, genetic sequencing data or genetic array data for a plurality of genetic positions, determining respective true ploidy state values for a plurality of genetic segments, each genetic segment respectively comprising at least some of the plurality of genetic positions, based on the genetic sequencing data or genetic array data, and determining a neural network comprising one or more layers for calling respective ploidy state values, the neural network defined at least in part by a plurality of weights. The method further includes iteratively modifying the neural network until an exit condition is satisfied, the modifying including determining a batch of data comprising a plurality of cases, each case corresponding to a respective genetic segment of the plurality of genetic segments and comprising data indicating an allele frequency for one or more positions of the respective genetic segment, generating a synthetic case based on one or more of the plurality of cases of the batch, and including the synthetic case in the batch to generate an augmented batch, augmenting the true state values based on the synthetic case, propagating the batch of data through the neural network to generate a network output comprising one or more respective state values for each case, and modifying one or more of the plurality of weights based on the loss values. The method yet further includes selecting a test sample comprising plasma extracted from a pregnant mother, and calling, for the test sample, a ploidy state for a target genetic region by propagating genetic sequencing data for the test sample or genetic array data for the test sample through the modified neural network.

In another aspect, the present disclosure provides a method of conducting pre-implantation genetic screening, including determining, for a training sample, genetic sequencing data or genetic array data for a plurality of genetic positions, determining respective true ploidy state values for a plurality of genetic segments, each genetic segment respectively comprising at least some of the plurality of genetic positions, based on the genetic sequencing data or genetic array data, and determining a neural network comprising one or more layers for calling respective ploidy state values, the neural network defined at least in part by a plurality of weights. The method further includes iteratively modifying the neural network until an exit condition is satisfied, the modifying including determining a batch of data comprising a plurality of cases, each case corresponding to a respective genetic segment of the plurality of genetic segments and comprising data indicating an allele frequency for one or more positions of the respective genetic segment, generating a synthetic case based on one or more of the plurality of cases of the batch, and including the synthetic case in the batch to generate an augmented batch, augmenting the true state values based on the synthetic case, propagating the batch of data through the neural network to generate a network output comprising one or more respective state values for each case, and modifying one or more of the plurality of weights based on the loss values. The model further includes selecting a test sample from an embryo, and calling, for the test sample, a ploidy state for a target genetic region by propagating genetic sequencing data for the test sample or genetic array data for the test sample through the modified neural network.

In another aspect, the present disclosure provides a method of calling a ploidy state using a neural network. The method includes determining, for a training sample, genetic sequencing data or genetic array data for a plurality of genetic positions, determining respective true ploidy state values for a plurality of genetic segments, each genetic segment respectively comprising at least some of the plurality of genetic positions, based on the genetic sequencing data or genetic array data, and determining a neural network comprising one or more layers for calling respective ploidy state values, the neural network defined at least in part by a plurality of weights. The method further includes iteratively modifying the neural network until an exit condition is satisfied, the modifying including determining a batch of data comprising a plurality of cases, each case corresponding to a respective genetic segment of the plurality of genetic segments and comprising data indicating an allele frequency for one or more positions of the respective genetic segment, propagating the batch of data through the neural network to generate a network output comprising one or more respective ploidy state values for each case, determining one or more loss values based on the one or more respective ploidy state values, using a loss function and the true ploidy state values, and modifying one or more of the plurality of weights based on the loss values. The method further includes calling, for a test sample, a ploidy state for a target genetic region by propagating genetic sequencing data for the test sample or genetic array data for the test sample through the modified neural network.

In another aspect, the present disclosure provides a method of training a neural network using augmented data, including determining, for a training sample, genetic sequencing data or genetic array data for a plurality of genetic positions, determining respective true state values for a plurality of genetic segments, each genetic segment respectively comprising at least some of the plurality of genetic positions, based on the genetic sequencing data or genetic array data, and determining a neural network comprising one or more layers for calling respective state values, the neural network defined at least in part by a plurality of weights. The method further includes iteratively modifying the neural network until an exit condition is satisfied, the modifying including determining a batch of data comprising a plurality of cases, each case corresponding to a respective genetic segment of the plurality of genetic segments and comprising data indicating an allele frequency for one or more positions of the respective genetic segment, generating a synthetic case based on one or more of the plurality of cases of the batch, and include the synthetic case in the batch, and propagating the batch of data through the neural network to generate a network output comprising one or more respective state values for each case. The method further includes modifying one or more of the plurality of weights based on the network output.

In further aspect, the present disclosure provides a system for training a neural network for calling a sub-chromosomal ploidy state including a processor and processor-executable instructions stored on non-transitory memory that, when executed by the processor, cause the processor to determine, for a training sample, genetic sequencing data or genetic array data for a plurality of genetic positions, and determine respective true state values for a plurality of genetic segments, each genetic segment respectively comprising at least some of the plurality of genetic positions, based on the genetic sequencing data or genetic array data. The processor-executable instructions, when executed by the processor, further cause the processor to determine a neural network comprising one or more layers for calling respective state values, the neural network defined at least in part by a plurality of weights, and iteratively modify the neural network until an exit condition is satisfied. The iterative modification includes determining a batch of data comprising a plurality of cases, each case corresponding to a respective genetic segment of the plurality of genetic segments and comprising data indicating an allele frequency for one or more positions of the respective genetic segment, selecting a portion of a first segment of a first case of the plurality of cases, selecting a second segment of a second case of the plurality of cases that has an aneuploidy based on the true state values, selecting a portion of the second segment, replacing the portion of the first segment with the portion of the second segment to generate a synthetic case, and including the synthetic case in the batch to generate an augmented batch, augmenting the true state values based on the synthetic case, propagating the batch of data through the neural network to generate a network output comprising one or more respective state values for each case, and modifying one or more of the plurality of weights based on the network output.

The foregoing general description and following description of the drawings and detailed description are by way of example and explanatory and are intended to provide further explanation of the implementations as claimed. Other objects, advantages, and novel features will be readily apparent to those skilled in the art from the following brief description of the drawings and detailed description.

### BRIEF DESCRIPTION OF THE DRAWINGS

The accompanying drawings are not intended to be drawn to scale. Like reference numbers and designations in the various drawings indicate like elements. For purposes of clarity, not every component may be labelled in every drawing.

FIG. 1 illustrates an overview of an example process for genotyping or sequencing a genomic or plasma sample, according to some embodiments.

FIG. 2 illustrates an overview of an example process of annotating the sequencing or array data, according to some embodiments.

FIG. 3 illustrates an example process of training a neural network, according to some embodiments.

FIG. 4 illustrates an example process of training a neural network, according to some embodiments.

FIG. 5 illustrates a detailed example of a neural network, according to some embodiments.

FIG. 6 illustrates an example of a classification network, according to some embodiments.

FIG. 7 illustrates an example algorithm for augmenting training data and truth data, according to some embodiments.

FIG. 8 illustrates an example algorithm for augmenting training data and truth data, according to some embodiments.

FIG. 9 illustrates an example of a neural network architecture, according to some embodiments.

FIG. 10 is a block diagram showing an embodiment of a ploidy calling system, according to some embodiments.

FIG. 11 is a flow chart illustrating an example method of calling a ploidy state for a target genetic region, according to some embodiments.

FIG. 12 is a flow chart illustrating an example method of modifying a neural network, according to some embodiments.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

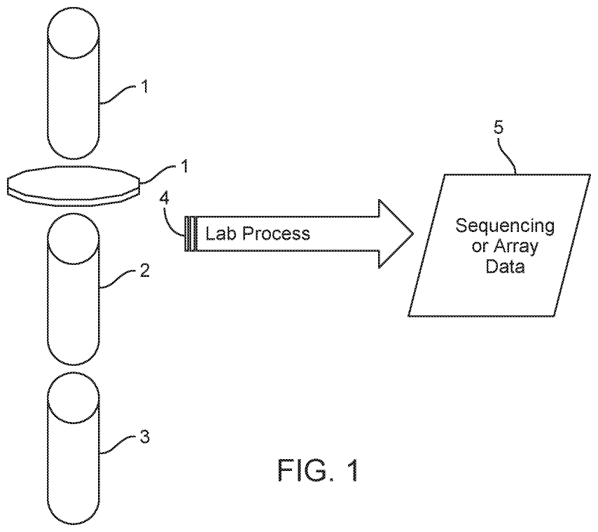

Referring now to FIG. 1, FIG. 1 shows an overview of an example process for genotyping or sequencing a genomic or plasma sample using, for example, a Cyto12b array or a targeted single nucleotide polymorphism (SNP) pool using Next Generation Sequencing (NGS). The Cyto12b array can have, for example, approximately 300 thousand (written here as ˜300 k) SNP targets across all chromosomes, and various NGS pools may, for example, have a smaller set of targeted SNPs ranging from hundreds of genomic positions to tens or hundreds of thousands of SNPs. The input into the sequencing or array genotyping process may include one or more cells from an embryo (1 in FIG. 1), as well as optional genomic samples from parents of the embryo (2 and 3 in FIG. 1). In some embodiments, the input into the sequencing process may be a plasma sample from a pregnant mother (1 in FIG. 1) (e.g. obtained by a non-invasive, with respect to the fetus, liquid biopsy). The output of the sequencing or array genotyping process, or lab process (4 in FIG. 1), after analytical processing, includes numerical array data (5 in FIG. 1) for each of the samples stored on some computer storage medium, which can include 2 or more numerical arrays of positive numbers per sample, where the length of each numerical array is equal to the number of genomic positions identified by the sequencing target pool or array and the individual entries in the numerical arrays represent counts or intensities per matching target position in the targeted pool of SNPs.

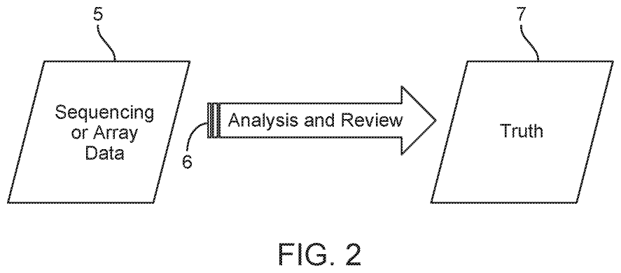

Referring now to FIG. 2, FIG. 2 shows an overview of an example process of annotating the sequencing or array data (5 in FIG. 2). For example, empirical and first principal algorithms in connection with visual hand review of the array data can be applied (6 in FIG. 2) to the output of the sequencing or array genotyping process. This can be done to classify the output data and obtain truth, or truth data (7 in FIG. 2) about the state of individual chromosomes, of the embryo or fetus, or of the plasma itself when sequencing a liquid biopsy for detecting cfDNA containing somatic variants possibly causing cancer or other disease in the individual. The truth data can be used as reference data, and may be assumed to indicate, for example, an accurate classification of an analyzed sample. The truth data can be stored on some computer storage media for training a neural network. This truth data may include a classification and a likelihood of each chromosome identified from the embryos or fetus as being in a euploid state, or one of a number of aneuploidy states. For a plasma sample used for detecting a disease, such as cancer, in the host individual, the truth data may contain match-normal data about genomic locations and description of germline variants from the individual obtained by sequencing a genomic sample, e.g., buffy coat from the liquid biopsy from which the plasma is obtained or obtained at a different time-point from the individual. In addition the truth data, when using a plasma sample to detect cancer, can contain information (e.g., quantification and/or location) about the somatic variants and/or other sub-chromosomal abnormalities associated with the cancer, and can be obtained by sequencing a cancer sample and comparing the results to the match-normal sequencing data or to publicly available reference genomic data for humans.

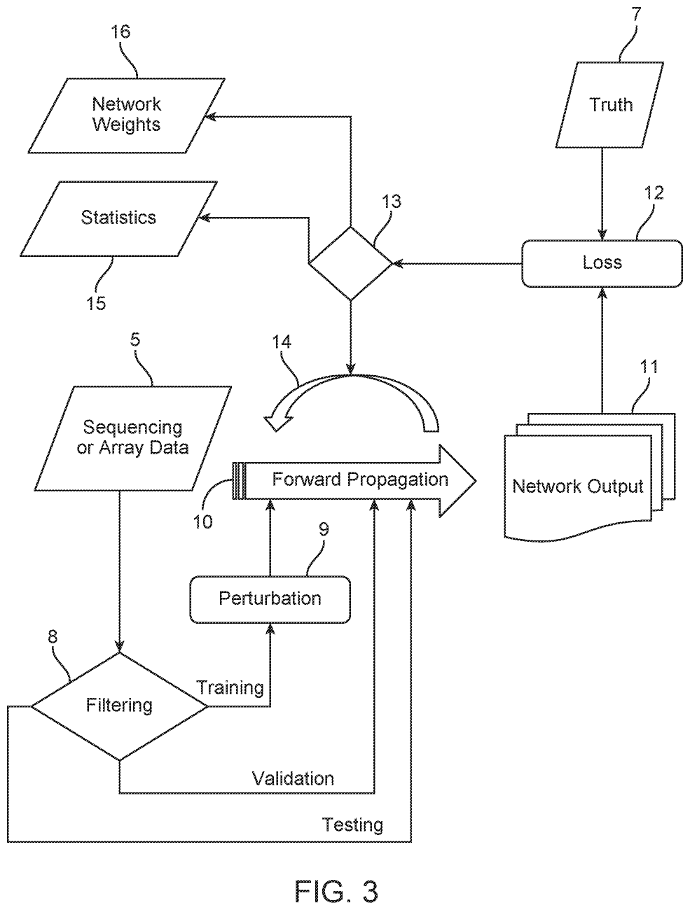

FIG. 3 shows an example process of training a neural network, which may be a deep neural network. The process uses the sequencing or array data 5 and the truth 7 as described with respect to FIGS. 1 and 2, to train and evaluate neural networks (e.g. to output array data and truth data), or to improve the truth data and the classification per chromosome or target genomic position.

In some embodiments, the sequencing or array data 5 is divided into groups by a filtering process 8. The groups include training data, validation data and testing data. Validation data and testing data can include data set aside for later testing on a trained neural network (e.g. the validation data can be used to test for overfitting during an optimization process, and the testing data can be used to quantify the predictive power of the final network). During training, the training data may be perturbed (9 in FIG. 3) to regularize the neural network, and to provide better generalization and to make the network resilient when it comes to additional noise and examples that are not part of the existing training set. The perturbing process 9 in FIG. 3 also may include computing additional derived attributes that are useful for training the network in order to minimize an output of a loss function (12). Data is fed through a forward propagation process (10 in FIG. 3) in batches to generate a network output (11 in FIG. 3) that can be compared to the truth (7) to compute one or more loss values (12 in FIG. 3), using the loss function. The loss values are functions of weights in the neural network and these weights may be optimized, updated, or otherwise modified to generate a new neural network output 11 closer to the truth (e.g. resulting in a lower loss value), over multiple iterations. Such an optimization process (14 in FIG. 3) modifies the weights of the network before a new batch of sequencing or array data is passed through the network. The optimization process can be a modified form of a stochastic gradient descent optimization, for example, or another appropriate optimization process. When an exit condition is reached (e.g. one or more loss values are determined to be below or equal to a predetermined threshold (e.g. a predetermined validation threshold)), the training process ends, and the network weights (16 in FIG. 3) are stored on computer readable media and can be deserialized to build a function that maps the sequencing or array data to an output according to the forward propagation function specified by the network. The training process may also create (e.g. using validation data and testing data) validation statistics (15 in FIG. 3) that can be used to guide the training process and unbiased testing statistics after the training is completed.

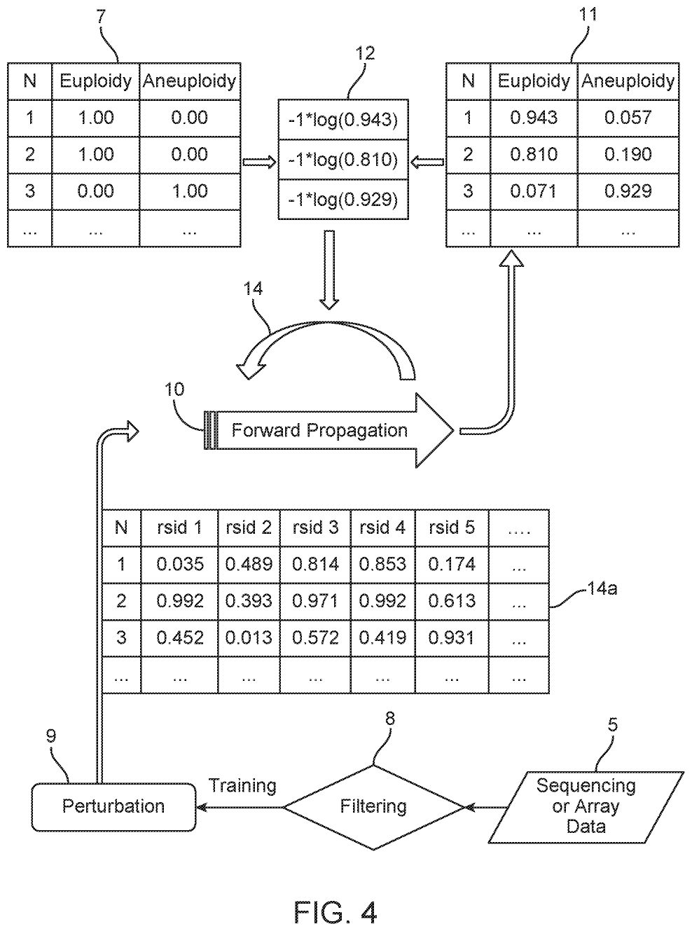

FIG. 4 shows an example implementation of a training phase for a neural network. The network can then, after training, be used to classify embryos as being in a euploid or an aneuploidy state by running sequencing or array numerical data through the same input pipeline and forward propagation process. The inputs into the network can include two or more (possibly normalized) numerical arrays that are the output of sequencing or array processes as described in connection with FIG. 1. An allele frequency (e.g. an allele ratio, which can be a ratio of a number of reads of an aneuploidy allele to a total number of reads, or an allele frequency) obtained for each of a set of samples (e.g. 1-3 samples (embryo or plasma and optional mother and father genomic samples)) may also be input into a first layer of the network. The allele ratios from the embryo or plasma may, in some embodiments, be the only input. FIG. 4 shows a matrix (14a) where each row contains the allele ratios from one embryo or plasma for data that has been selected as training data at process (8) and parsed, transformed and perturbed in process (9). The columns represent genomic positions. When working with cells from an embryo biopsy, embryo allele ratios may be input, as shown, and in some embodiments the allele ratios for three samples (embryonic, maternal, and paternal samples) are input. When working with plasma from a liquid biopsy of a pregnant woman, the normalized sequencing or array data reads or intensities and allele ratios from the plasma may be input. When working with plasma from a liquid biopsy of an individual that may have or may have had cancer, when the object is to train the network to quantify cfDNA, e.g., somatic variants, from the cancer present in the plasma, the input channels can, for example, include sequencing data from a match-normal sample, locating at least some of the germline variants of the individual, obtained, for example, by sequencing the buffy coat material obtained from the liquid biopsy (e.g., a blood sample). The input may also contain data about the somatic variants identified in a current or earlier cancer sample obtained from the individual if such a sample is available. This can be in addition to the channels inputted with high depth-of-read (ref and mut) sequencing of the plasma itself. Matrix (14a) is an example of one training batch that includes a number of “examples” (also referred to herein as “cases”), that may be randomly chosen from a pool of examples. FIG. 4 also shows an example network output (11) as described in FIG. 3, the truth data (7) and the loss values (12), which can be determined based on the truth data (7) and the network output (11). One example process includes computing the loss values (12) using a loss formula, such as a cross-entropy formula. A neural network can accept as input the array data obtained from the embryo, mother and father samples. The network can include trainable variables that can be used to modify the network output during the optimization process (14). The network output (11), is, for example a classification vector such as (x,y) with x and y numerical non-negative values that sum to 1 and where x>>y indicates a euploid classification and y>>x indicates an aneuploid classification of the embryo. In the case of training a classification network to detect the presence of somatic variants associated with cancer in a plasma sample, y>>x can, for example, indicate that the network detected presence of such variants and x>>y can indicate that the network did not detect the presence of the somatic variants. For example, if the x value is greater than the y value by a predetermined amount (which may, in some embodiments, be zero, or a negative amount), the system may classify the sample as euploid, and if the y value is greater than the x value by a predetermined amount (which may, in some embodiments, be zero, or a negative amount), the system may classify the sample as exhibiting aneuploidy. Each row shown in the network output (11) represents the output of such a vector for each of the input rows of the matrix (14a). The number of states, equal to the number of columns in matrices (7) and (11) in FIG. 4 (e.g. two states), depends on the available states of the truth data used to train the network. The output of the network may also be a single value that is approximated using a different loss function such as absolute difference to the truth value (L1 norm) or distance squared (L2 norm). An example of such a value is the fetal fraction found in a pregnant mother's plasma. Another example is the quantification of DNA from somatic variants associated with cancer in a plasma sample from the host. The loss values (12) for a batch may be defined as the average or sum of the individual losses for each example included in the batch. Any other appropriate loss function may also be used.

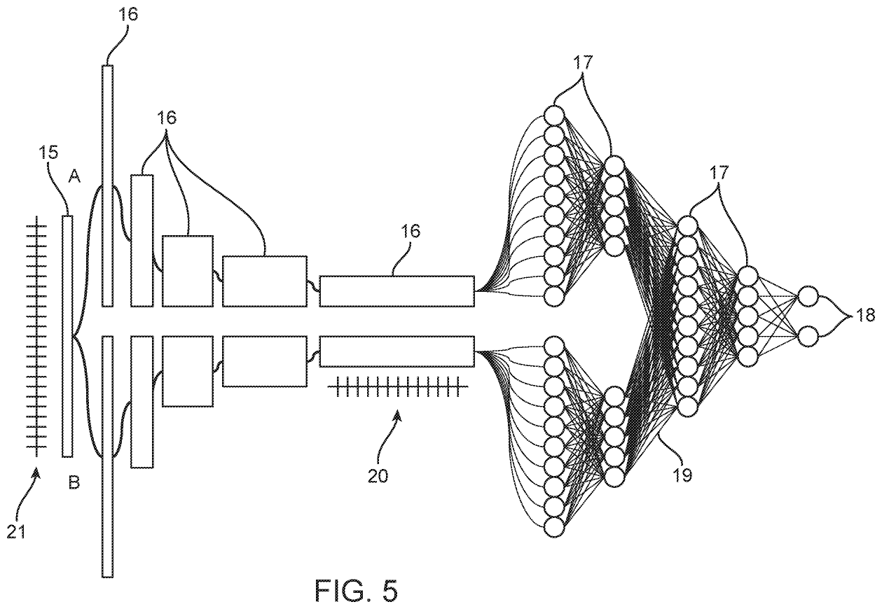

FIG. 5 shows a detailed example of a neural network as described in FIG. 3 and FIG. 4 that can be used for training (e.g. using stochastic gradient descent-like optimization) and then used to classify the state of an embryo or fetus chromosome using a forward pass process. The network starts with an input (15 in FIG. 5) of an N by 3 by ˜300 k numerical tensor, where N is the number of examples being classified together or batched during training when working with the Cyto12b array, the 3 channels are embryo, mother and father allele ratios, and the final number ˜300 k represents the number of genomic locations being targeted (21 in FIG. 5). In case of working with plasma, in some embodiments, an input (15 in FIG. 5) of N by 5 by ˜12 k, where again N is the number of examples batched together, ˜12 k is the number of genomic locations (21 in FIG. 5) and the 5 channels are the allele ratios for the plasma and four (e.g. normalized) output arrays from the NGS sequencing process such as reference allele reads, mutation allele reads, quality score and allele read error rates. The genomic locations don't have to apply to all the input channels since some of the input channels may be reordered according to different criteria. The plasma setup described below also includes a setup of just having one input channel instead of 5 (e.g. the plasma allele reads), and a number of other combinations are possible. The process can include a plurality of series (A and B in the depicted example) within the network, which may be fed different input tensors, some indexed by genomic location and some not. The network shown includes multiple initial one-dimensional convolutional, activation and pooling layers, denoted as 16 in FIG. 5, that reduce the size of the input vector, and extract relevant features in the form of additional channels (exemplified by 20 in FIG. 5). The input (15) can be channelled to multiple such series of convolutional layers that include multiple pooling and activation functions. FIG. 5 shows examples of two such series denoted by A and B in the figure. The series of multiple layers may also be chained together. The series of layers then extends to one or more series of fully connected layers (17 in FIG. 5), with dropout and other regularization techniques optionally embedded. The fully connected layers may have hundreds or thousands of nodes resulting in millions of weights (19 in FIG. 5) between the nodes. The fully connected layers are then concatenated together and eventually lead to a final logits layer (18 in FIG. 5) of size N by k where k is the number of classes in the classification desired, for example, as shown (18) where k=2 representing two classes: euploidy state and aneuploidy state. The final output (18) can, in some embodiments, be a single variable intended to indicate a statistical quantity such as the fetal fraction in the mother's plasma when such quantities are available in the truth set. During training and use for classification, the logits (18) may be fed into a softmax calculator to obtain confidence values for each state and during training a loss function is applied such as cross-entropy (see loss values 12 in FIG. 4 and FIG. 3), before computing the gradient with respect to the weights used in the network.

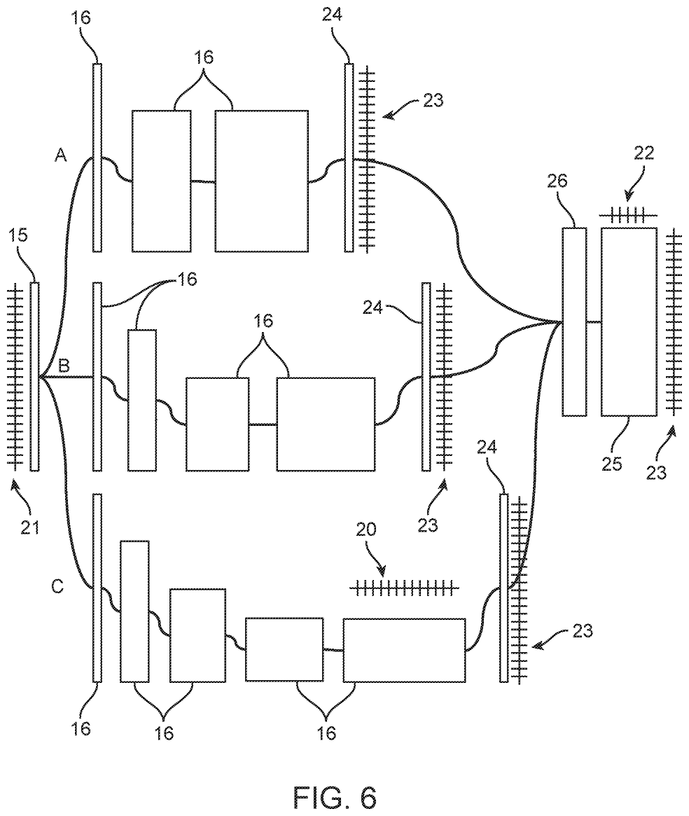

FIG. 6 shows an example of a classification network where the network outputs one set of classes per genomic location (23 in FIG. 6). The classes represent the state of the embryo or fetus at the given genomic target or SNP. For example, a set of 5 classes would be represented by a final convolutional layer (25 in FIG. 6) having 5 channels (22 in FIG. 6) each representing one of the logits used for computing the likelihood of, for example, maternal monosomy, paternal monosomy, disomy, maternal trisomy or paternal trisomy at each genomic position or genomic bins, as exemplified by the axis shown (23 in FIG. 6). In this case the input is of the same type as exemplified in FIGS. 5 (15 and 21) but the output layer includes N by “number of genomic positions” (23 in FIG. 6) by k (22 in FIG. 6) tensor where each final dimension of k channels represents the k classes representing the truth states (7) obtained and explained in connection with FIG. 3 and N is the number of examples being classified together or batched together during training, validation or testing phase. The network may include multiple one-dimensional convolutional layers, activation and pooling layers (16 in FIG. 6) followed by one or more transpose convolutional layers (24 in FIG. 6), also referred to as a deconvolution layer, as well as optional layers used for smoothing the output (26 in FIG. 6) and the last convolutional layer (25 in FIG. 6). The training and optimization proceeds using, for example, mini-batch gradient descent and momentum type optimization such as the Adam optimization algorithm. FIG. 6 shows several series of the convolutional-deconvolutional setup (A,B,C in FIG. 6). Each of the series ending in the corresponding deconvolutional layer (24 in FIG. 6) can optionally be trained individually using respective loss functions, and other weights in the network (e.g. from additional convolutional layers such as layers (26) and (25) in FIG. 6) can then be trained using the input from the deconvolutional channels as input channels.

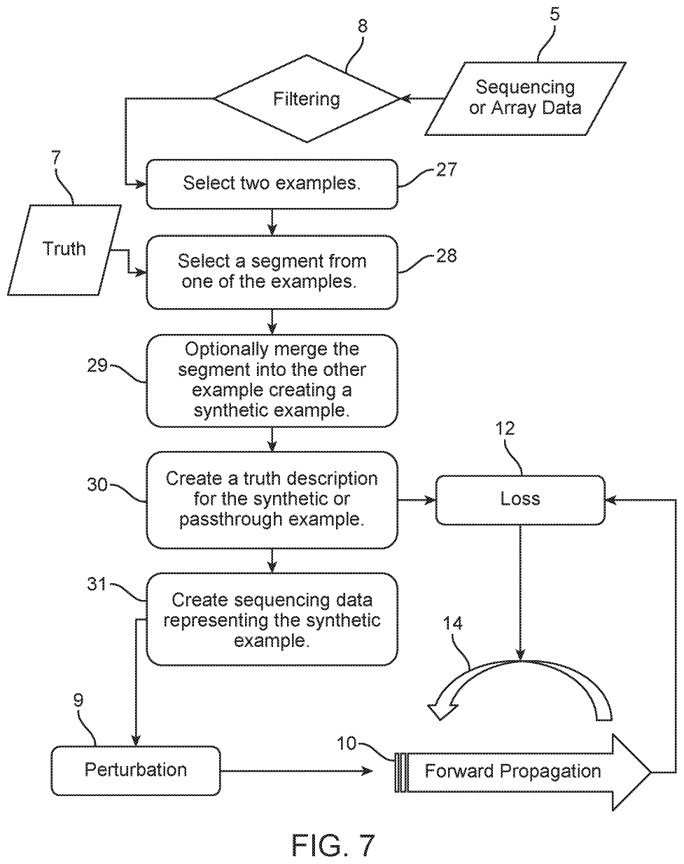

FIG. 7 shows an algorithm for augmenting the training data and truth data in such a way that after training of the neural networks (e.g. as illustrated in FIGS. 3, 4, 5 and 6) the networks are able to classify segments of chromosomes as being in euploid or one of a plurality of aneuploid states. For the neural network show in FIG. 5 the network, using the augmented truth and sequencing or array data set, is trained to detect the state of the embryo as having a segmented or whole chromosome aneuploidy by the augmented dataset shown. The neural network shown in FIG. 6 is trained to detect and locate the SNPs or genomic positions, within the embryo's or the fetus's genome that are in various ploidy states based on the augmented training set. Sequencing or array data and truth data is augmented during training as shown in FIG. 7 using one or more synthetic cases or examples. To generate a synthetic example the algorithm selects (27 in FIG. 7) two examples from the training set. This can be done randomly, and one of the examples (e.g. the second example) is picked from the training set so that it is guaranteed, by the truth data, to have a whole chromosome or regional aneuploidy. For example, the system can determine that the second example has a whole chromosome or regional aneuploidy, and can select the second example based on that determination. The algorithm selects (e.g. randomly) a segment, which may be of some minimum length, within the aneuploidy region (28 in FIG. 7) of the second example and replaces, process (29 in FIG. 7), the corresponding sequencing or array data from the first example by the data from the second example. The data replaced from the first example by data from the second example may correspond to the genomic positions from the aneuploidy segment selected from the second example. Process (29 in FIG. 7) may selectively (e.g. randomly or based on other criteria) pass the first example unchanged through the system so that during training the network may also be trained using unaltered examples. In the next process (30 in FIG. 7) shown, the algorithm modifies the truth data submitted to the loss computations so that the inserted segment is counted as an aneuploidy segment in the modified first example when the example is submitted, process (31 in FIG. 7), as part of a larger batch containing a mixture of synthetic and unaltered examples to the neural network during the training phase of the network, as described above in connection with FIGS. 3 and 4. During the selection process (27 in FIG. 7), examples are selected so that the sequencing or array data statistics found in the truth set or otherwise computed for the two examples is similar within a set range. In case of plasma from a pregnant mother this would include the two examples selected for producing the synthetic sequencing or array data possibly having a similar fetal fraction statistics. During training this procedure is repeated again during each epoch or cycle.

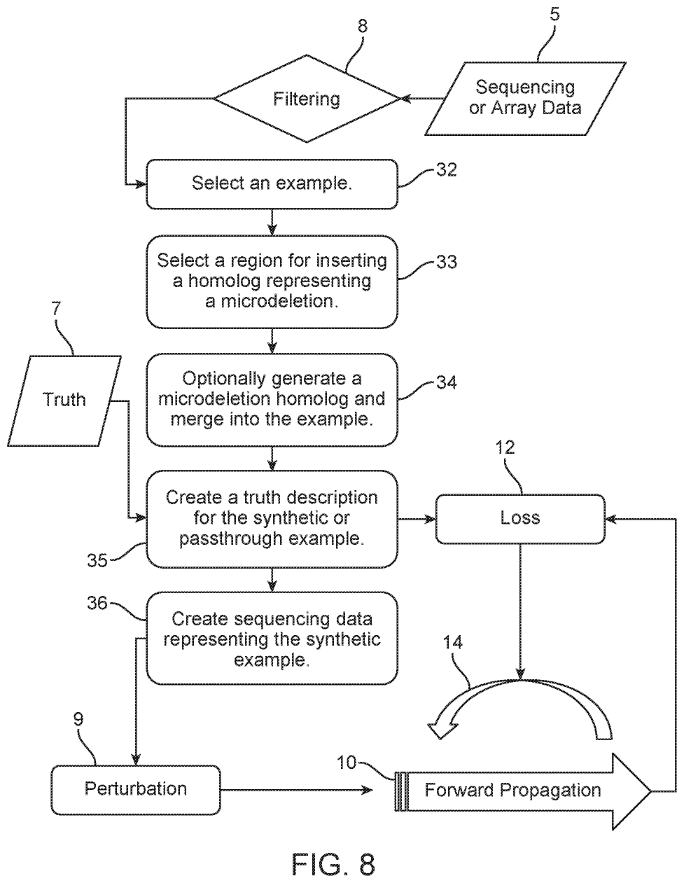

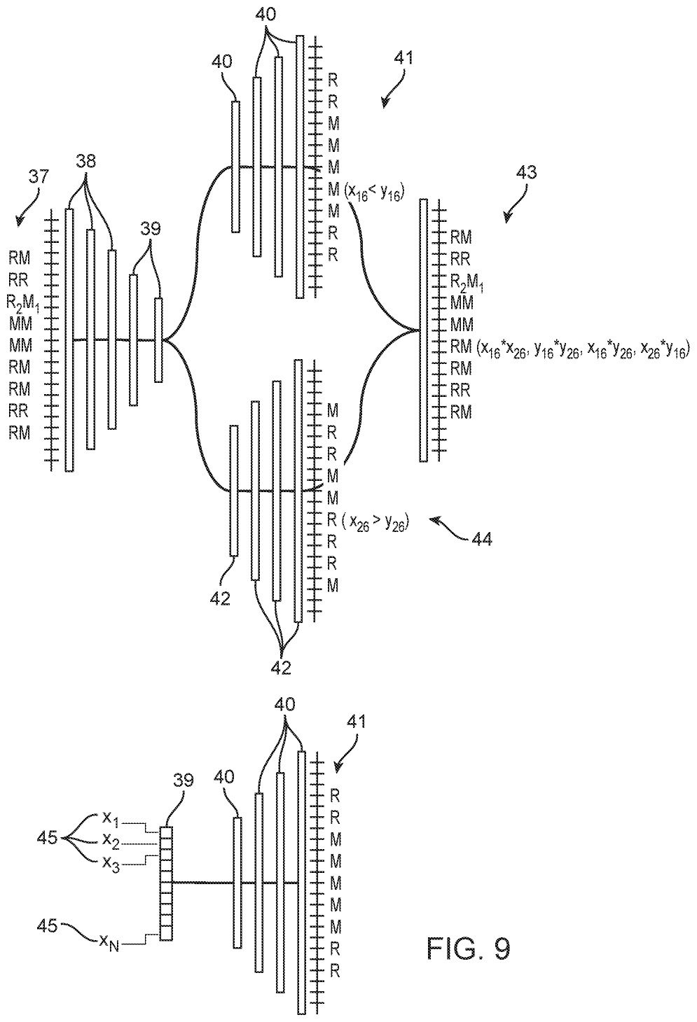

FIG. 8 shows an algorithm for augmenting the training data and truth data by inserting synthetic sequencing or array data (e.g., allele reads), representing small chromosomal deletions in various regions of the chromosome, such as where such deletions are known to take place and cause known conditions. The trained network using this augmented data learns to classify these regions based on the existence of the deletions. Different types of networks, such as those shown in FIG. 4, 5 or 6 can be trained using this augmented data resulting in both a classification algorithm and a more general deletion location algorithm. The algorithm assumes that during training of a neural network with the ability to detect small chromosomal homolog deletions (e.g., microdeletions) in predefined regions of the genome the following procedure can be used. The first process is to select examples from the training set (32 in FIG. 8) and selecting, for each example selected, a region (33 in FIG. 8) (e.g. from a list of predefined microdeletion regions representing known conditions). The microdeletion regions could, for example, include one or more of the regions associated with the following genetic conditions and diseases: 1p36 Deletion, 1q21.1 Distal Microdeletion, 2q37 Microdeletion: Albright Hereditary Osteodystrophy-like/Brachydactyly, 3q29 Microdeletion, Wolf-Hirschhorn syndrome, Cri Du Chat, 5p15.2 Microdeletion, William-Beuren Syndrome, Langer-Giedion/Trichorhinophalangeal type II, 9q34 Microdeletion/Kleefstra Syndrome, 10p13-p14 DiGeorge 2, 11p13 Microdeletion: WAGR, 11q24.1 Microdeletion: Jacobsen Syndrome, Angelman, Angelman Syndrome Type 2, Prader-Willi Syndrome Type 2, Prader-Willi, 16p11.2 Microdeletion, 16pter-p13.3 Microdeletion: AT-ID, Smith Magenis, Miller Dieker Syndrome, RCAD (17q12 del), 17q21.31 Microdeletion, 18q21.2 Microdeletion: Pitt-Hopkins Syndrome, DiGeorge, 22q11.21 Microdeletion, 22q11.2 Microdeletion, Phelan McDermid 22q13 Deletion, 5q22 Microdeletion: Familial Adenomatous Polyposis with ID, 5q35.2-35.3 Microdeletion-Sotos Syndrome, 6p25.3 (p24) Microdeletion, 8p23.1 Microdeletion CDH2, 11p11.2 Microdeletion: Potocki-Shaffer Syndrome, 13q14.2 Deletion, Retinoblastoma with ID, 13q32 Deletion-HPE5, PKD1/TSC2 Contiguous Deletion Syndrome, 17p13.3 Distal Microdeletion, 17p13.3 Distal Microdeletion, 17q21.31 Microdeletion, Isochromosome, 21q22.3 Microdeletion: Holoprosencephaly 1, Pelizaeus Merzbacher XL. The region selected may be altered in size and position within a set range. In a homolog generating process (34 in FIG. 8), the algorithm generates, with a predefined frequency, a simulation of the sequencing or array data representing a microdeletion case in the region selected and optionally replaces the existing data from the genomic locations selected with the simulated data taking into account statistics such as the fetal fraction and the fetal DNA distribution in the case of mother's plasma. The inserted microdeletion data may come from actual known cases of such a preselected condition or it may be generated by a second neural network as described in connection with FIG. 9 herein, or the second neural network described below. In a truth generating or updating process (35 in FIG. 8), the truth data is modified and passed to the neural network to accurately represent the microdeletion or passthrough case. A process of generating sequencing data representing the synthetic example (36 in FIG. 8) may be implemented, and the generated sequencing data for the synthetic example can be perturbed and passed forward for propagation through the neural network.

Some embodiments implement a second neural network, and may implement a method using Generative Adversarial Networks (GANs) to train a neural network to generate individual homolog segments representing the population occurrence of these segments. The GANS may include a generative network and a discriminative network. The generative network may include two (e.g. identical) homolog generative networks, each of which produce single segment homologs. The output of the generative network is unphased segment genotypes produced by combining the two homologs produced by the two homolog generative networks. The discriminative network distinguishes the unphased genotypes produced by the generative network from real unphased genotype data. To train the GAN, the discriminative network is trained to distinguish unphased genotypes produced by the generative network from real unphased genotype data, and the generative network is trained to “fool” the discriminative network (to produce unphased genotypes that the discriminative network cannot distinguish (or has difficulty distinguishing) from the real unphased genotype data). Once trained, the generative network can be used to generate statistics for the homologs used to create synthetic data, and to augment and replace part of the training data as explained in connection with FIG. 8, and thereby enable the neural networks described above to detect related chromosomal abnormalities including microdeletions causing serious conditions in a fetus or embryo.

FIG. 9 shows a schematic neural network architecture (e.g. for a second neural network) that can be trained to generate individual homolog segments (41 in FIG. 9) representing the population occurrence of these segments. The network is related to a group of deep neural networks called autoencoders. The input (37 in FIG. 9) into the network for training is an unphased set, and randomly or otherwise selected phased genotypes, of the genotypes compatible with a subset of the genomic locations used and available as part of the population sequencing or array data (5). The generated statistics for the homologs is used to augment and replace part of the training data as explained in connection with FIG. 8 and thereby enable the neural networks described earlier to detect related chromosomal abnormalities including microdeletions causing serious conditions in the fetus or embryo. Multiple types of networks can be used to represent the encoder (38 in FIG. 9) and decoders (40 and 42 in FIG. 9). These include convolutional layers with pooling and activation functions for encoding or fully connected layers with dropout and activation functions for encoding and transpose convolution and convolution for the decoding layers or fully connected layers with dropout and activation for the decoders. Various technologies for creating autoencoders may be implemented, and some are explained in connection with FIG. 6.

Description of some embodiments follow. This description is provided by way of example only, and other embodiments consistent with the methods and systems described herein are encompassed by the present disclosure.

Some embodiments of applying the network shown in FIG. 5 to array data from genomic samples of only few cells are described below. The network in FIG. 5. is trained using a training subset of over 80,000 samples of array data from, approximately, embryo biopsies (e.g. 5 day embryo biopsies) performed during IVF cycles, blood samples from the embryo's parents and labelled algorithm generated and hand reviewed truth. For each example the input includes 3 channels one for embryo allele ratios, one for mother allele ratios and the third for father allele ratios all genotyped using the Cyto12b array at about 300,000 genomic locations for each of the 3 samples, spanning all the chromosomes. The allele ratios are the ratios x/(x+y) at each array SNP location where x and y are the 2 array channel intensities generated by the array genotyping process. The hand labelled embryo whole chromosomal state truth is available per embryo chromosome and is used to classify the embryo as being euploid or in an aneuploid state. Following the input layer some embodiments uses about 10 convolutional layers following two distinct paths or series as shown in FIG. 5, as series A and B. Each of the convolutional layers is followed by an activation “elu” function and a max pool layer. The first set of the convolutional and max pool layers start by expanding the number of channels from 3 to 16 each and scan a region of 512 and 1 consecutive locations respectively before performing a max scan of 256 consecutive location on the activation function's output followed by a max pool with a shift of 16. This structure is then repeated about four more times, for each series A and B, with different scan and max pool sizes each time doubling the number of output channels in each process. The scan sizes for some embodiments follows a pattern of 32, 16, 8, 8 for each of the series A and B in FIG. 5 and a pattern of 16, 8, 4, 4 for the max pool of each of the layers in the series after the first layer in each series. Following each of the series of convolutional layers, fully connected layers are added with 1024 followed by 256 nodes and then some embodiments concatenate the fully connected layers and adds two more additional layers of size 128 and 2 or some number equal to the number of ploidy states being sought and available in the truth set. The two nodes in the final layer simply represents the two classes “euploid” and “aneuploid”. Some embodiments implement a dropout rate between about 25% and about 75% for each of the fully connected layers except the final layer and each of the fully connected layers except the last is followed by the elu activation function. The associated input pipeline, shown in FIG. 3 and FIG. 4 applies perturbations to the input data including, for example: randomly permuting the array reads per SNP, randomly switching the role of the mother and father samples for the autosomal reads and perturbing the array reads randomly by multiplying them with scalars drawn from a distribution with mean close to 1 and a relatively small standard deviation. The training of the neural network proceeds and is serialized based on specified criteria when met by a validation sample set. Some embodiments use a stochastic gradient descent-like algorithm with momentum called Adam, and sets the learning rate to about 0.0001 and uses a batch size of 32.

Some embodiments for detecting sub-chromosomal aneuploidies adapt the network shown in FIG. 5, and described above, to detect sub-chromosomal segments of aneuploidies such as deletion segments, duplication and/or trisomy segments by applying the algorithms shown in FIG. 7 or the algorithm shown in FIG. 8 to the input pipeline of FIG. 5. This process can include locating in the truth data (see 7 in FIG. 2, FIG. 3, FIG. 4, FIG. 7) one or more samples of such aneuploidies from other examples known to contain whole chromosomal aneuploidies by the truth labelling. The selection can be done to examples randomly during training with a predetermined frequency. For example, the selection can be done with a frequency of 50% or more, or 33% or more. In some embodiments, the frequency is between 25% and 66%. An array segment of some minimum length (e.g. at least 100 SNPs), is then copied from the one or more randomly selected aneuploidy chromosome data (x and y intensity reads, or the allele ratios directly) starting at a random location and inserted into the examples being processed for training as indicated in FIG. 7 (process 29). Corresponding segments from the father and mother array data of the selected random example are also inserted into the father and mother array data, respectively, for the training example. The label used for the training example is modified (e.g. temporarily) during training to represent the changed truth state of the modified example as indicated by the descriptive workflow outlined in FIG. 7, or a similar workflow for detecting microdeletions shown in FIG. 8. The resulting neural network after successful training will be readily able to detect sub-chromosomal aneuploidy segments when new data is passed through the network using forward propagation, to harness the network for classification.

In some embodiments, sequencing data obtained from targeted Next Generation Sequencing when sequencing plasma from pregnant mothers and a smaller target set (genomic locations) of approximately 13,000 SNPs from regions includes, for example, chromosomes 13, 18, 21 and chromosome X, and some embodiments of the network shown in FIG. 5 use a similar and scaled down structure in terms of convolutional kernel sizes, so that the initial convolutional network will employ a kernel of 128 genomic positions, 4 input channels, 16 output channels, a max pool over 64 locations with a max shift of 16 locations. Following this, some embodiments employ additional layers (e.g. about five additional layers) of convolution, activation and max pool before switching or flowing to fully connected layers. Some embodiments can employ a high dropout rate (e.g. about 65% or more, about 75% or more, about 85% or more, or higher), in the fully connected layers, and can implement a linear bottleneck layer to avoid overfitting. The rate of aneuploidy labels in the training set may be low, for example, between one and two percent, so in addition to the techniques described above in connection with array data, including adding noise, perturbing the reads and switching the role of the reference and mutation reads, some embodiments include relabelling examples after having replaced and permuted parts of the training data in a given example with data from a chromosome of a different example having an aneuploidy and a similar plasma fetal fraction, as determined by the truth data, and include following the processes shown in FIG. 7 or FIG. 8. In some embodiments, in some implementations of whole chromosome aneuploidy calling, a minimum number of SNPs in process 29 in FIG. 7 is used (e.g. a number based on, and/or close to (e.g. +/−5%), the number of locations on a given chromosome and a maximum length equal to the number of available SNPs on the given chromosome). Some embodiments implement a target learning rate of about 0.0001 as well as a learning rate schedule, a mini-batch size of about 128 and a reduced weight of about 0.25 for the aneuploidy examples in addition to increasing their frequency in the training batches.

In some natural network topology embodiments, referred to herein as bias model for reads, used when classifying plasma from pregnant mothers, includes starting with the reference and mutation plasma reads from approximately 13,000 genomic locations from chromosomes 13, 18, 21 and X. The embodiment may include reads from additional or fewer chromosomes. The reference and mutation reads start out as two initial channels or features from the processed or aggregated Next Generation Sequencing reads (“ref” and “mut” reads) as input into the network and then building a series of convolutional layers increasing the number of channels or features, but keeping the scan length to one genomic location, from 2 to 128 channels, from 128 to 64, from 64 to 32, from 32 to 16, from 8 to 4, from 4 to 2 channels with each of the layers having a kernel of trainable weights and one traininable bias variable per feature and an elu activation function between each layer. The network then continues and employs a convolutional layer from 2 to 1 channels followed by the activation function, but in this case in addition to the one channel bias variable each genomic position, corresponding to the output of the network at this level, gets a separate trainable variable per outputted genomic position, sometimes called untied biases. After the model employs this particular model of tied and untied biases, the output data is again taken through a series of convolutions and activation functions changing the number of channels or featuresfrom 1 to 128, from 128 to 64, from 64 to 32, from 32 to 16 and from 16 to 8 each time including a feature bias per channel and followed by the elu activation function and a scan size of 1. The size of each network layer is then modified by adding 6 more convolutional layers employing only tied feature biases and followed by the activation function and max pool layers each. The scan sizes for these six layers are 128 for the first of the six layers and then each layer has a scan kernel of size 4, the number of channels is doubled by each layer, max scan is set at 64 and 8 for the first two layers and then fixed at 4 and max pool or shift is set at 16, 8, 4, 4, 2 and 2 for the respective 6 final convolution max pool layers. Following all these convolutional layers two fully connected layers, and elu activation, with dropout are used, the first one with 1024 nodes and the second one with 256 node and a high dropout rate of over 90% may be used, depending on the processing of the input data and how the positive cases are repeated multiple times either by insertion (see FIG. 7) or by artificially increasing their frequency in the training set by repetition and/or weight. Finally a linear logits layer with 2 outputs is attached in order to obtain the classification results as described in connection with FIG. 5. The training process may then proceed as described herein.

For sub-chromosomal aneuploidy calling when using targeted Next Generation Sequencing plasma sequencing, some embodiments implement the algorithms shown in FIG. 7 using a small minimum number of SNPs for processes 28 and 29 in FIG. 7. Some embodiments employ the algorithm shown in FIG. 8 for a specific microdeletion using mixed-in synthetic population data generated using decoder networks 40 and 42 in FIG. 9 for process 34 in the algorithm. The merged segments are selected at process 29 in FIG. 7 as, for example, continuous segments with start positions selected using a stochastic process (e.g. random start positions) and length from whole chromosomal aneuploidies coming from plasma data with similar fetal fraction for both the training example at hand and the example containing the given aneuploidy sample as described further in FIG. 7.

For locating, up to SNP level resolution, sub-chromosomal segments of aneuploidies within the various chromosomes some embodiments use a segmentation network shown in FIG. 6. Some embodiments include three different paths or series shown as A, B, C in FIG. 6 and as explained above in connection with FIG. 6. For array data, some embodiments use convolutional layers followed by a ReLu activation function and max pool for compressing the data. Layers A, B and C in some embodiments start with one convolutional layer with 3 input channels (embryo, mother and father allele ratios for each genomic location), a scan size of 512 consecutive locations and 32 output channels, followed by the activation function and a max scan of 256 consecutive genomic locations and a max pool step size of 32 before adding two more convolutional layers, each including an activation function, increasing the channels from 32 to 64 and then to 128, each with a scan of 8. Some embodiments employ a transpose convolutional layer (24 in FIG. 6) with an output scan of 256, a stride of 32 and 2 output layers for path A. Following path B, some embodiments include at least one additional convolutional layer, with a scan length of 32 and doubling the output channels, followed by the activation function and a max pool layer with max scan of 16 and step size of 4. Path C employs yet another convolutional layer with a scan length of 16 and again doubling the output channels, followed by the activation function and a max pool layer with max scan of 8 and step size of 4 as shown by the layout in FIG. 6. For paths A and B, some embodiments employ similar convolutional layers following the last max pool layers as for path C, but with adjusted channel input and output numbers and as before with a ratio of 2 for the channel numbers in each process as before. The transpose convolutional layer (24 in FIG. 6) following path B has a stride length of 128, output scan of 256 and reduces the number of channel to 2. The transpose convolutional layer (24 in FIG. 6) following path C has a stride length of 512, output scan of 256 and reduces the number of channel again to 2.

The 6 output channels, 2 each from the 3 transpose convolutional layers, are then combined into 6 channels and passed through two more convolutional layers each followed by a ReLu activation function. The final layer in some embodiments has 2 final output channels, that are, after training, configured to distinguish between the euploid and aneuploid classes of each genomic location (SNP) by providing a confidence likelihood (e.g. a softmax confidence likelihood) of the genomic location belonging to a segment in each of the truth states, when supplied with unseen or non-annotated examples and using forward propagation and as described further in connection with FIG. 6 above.

For next generation sequencing data some embodiments implement input channels representing quantities such as allele ratios from the mothers plasma, normalized and scaled total number of reads per genomic location and one or more permuted set of the allele ratios. The segmentation network (e.g. as shown in FIG. 6) is scaled to match the size of the data (number of SNPs). In both cases the array data and the sequencing data goes through perturbations as described in connection with FIGS. 3, 4, and 5 above. In order to train the network to detect sub-chromosomal aneuploidies the algorithms shown in FIG. 7 and/or FIG. 8 can be included in the input pipeline, resulting in a system configured to locate sub-chromosomal aneuploidies in a way similar to the way that has been described above with reference to the array data. Some embodiments use a small minimum segment length in process 28 when training the network to detect sub-chromosomal aneuploidies.

Some embodiments use the trained neural network shown in FIG. 9 to create decoding subnetworks, shown as subnetworks 40 and 42 in FIG. 9, that are used to generate sequencing or array data used in process 34 of the training algorithm shown in FIG. 8. Some embodiments of the network shown in FIG. 9 use an input layer, 37 in FIG. 9, corresponding to approximately 1000 SNPs focused on a specific genomic region of the genome. The classes inputted into the initial convolutional, activation and max pool layer at each location are genotypes represented as 4 channels shown as a vector of size 4 and explained below. The randomly (or otherwise) selected phased heterozygous genotypes can be used to determine which of the two parental decoder subnetworks (40 in FIG. 9 or 42 in FIG. 9) should output which homolog for each example. This network is trained to output (43 in FIG. 9) the same genomic sequence as inputted, so truth is known and the loss function is easily computed as a cross entropy function on the outputted softmax probabilities when training this network on a mini-batch of 128 examples. Following the first input convolutional layer, the number of channels is slowly increased in subsequent convolutional layers each of which is followed by an activation and max pool layer resulting in multiple encoding or compression layers as shown in FIG. 9 as structures 38 and 39. Some embodiments ensure that the number of input variables in the final decoding layer 39 greatly reduces, by the aggregation and max pool provided by the first layers, the number of input variables used in the beginning layer shown as 37 in FIG. 9. Following the last decoder layer, 39 in FIG. 9, two series 40 and 42 in FIG. 9 of transpose convolutional layers are employed in some embodiments to construct parental 1 (first parental) and parental 2 (second parental) homologs of having a length about equal to the number of genomic locations that are input (37), but with 2 channels each instead of the 4 channels employed for the input shown as 37. In order to generate the final output 43 in FIG. 9 a formula, explained below, is applied to the output of layers 40 and 42 in FIG. 9. The following processes can be used for connecting the genotypes between the input layer 37 in FIG. 9 and the outputs of the two subnetwork 41 and 44 of decoding networks 40 and 42, and the final output 43. For some embodiments the network structure is such that the two chromosomal homologs are represented internally in the network structure, as already explained, and the network may be subdivided to selectively output the generated homologs individually after training. The 5 genomic genotypes inputted per genomic location are the unordered (unphased) RR, RM, MM and the phased R₁M₂, R₂M₁ symbols found in population data at each input location for each example. The last two phased genotype classes R₁M₂, R₂M₁ represent respectively R (reference, genotype, allele or SNP at a given location) from parent 1 (40 in FIG. 9), M (mutation, genotype, allele or SNP at a given location) from parent 2 (network 44 in FIG. 9) and vice versa. Phased population sequencing or array data may thus be mixed in during training with the unphased data using the phased heterozygous genotypes. In order to accommodate the mix of phased and unphased genotypes the network can start with an input layer of 4 channels per genomic position where each position has attributes according to genotype as RR=(1,0,0,0), MM=(0,1,0,0), RM=(0,0,0.5,0.5), R₁M₂=(0,0,1,0) and R₂M₁=(0,0,0,1). Clearly, other representations are possible including permutations of the channels. The output of each of the decoder layers (41 and 44 in FIG. 9) is the likelihood vector (x,y) per genomic position with x>y representing R and x<y representing M for the genomic homolog position. The final output (43 in FIG. 9) is simply a function of the output from the decoder layers that maps the output from decoder layer for parent 1 (41) (x1,y1), and the output for parent 2 (44) (x2,y2) to the genotype likelihood value (x1*x2, y1*y2, x1*y2, x2*y1) representing the output channel values for each of the genomic positions included in the network's output (43). This operation may be applied before or after the softmax formulation and depending on the approach the formula is modified accordingly. FIG. 9 exemplifies this mapping by showing the formula for genomic position 6 on the FIGS. 41,44 and 43 in FIG. 9).

After the network shown in FIG. 9 has been trained using population array or sequencing data for the microdeletion genomic region at hand as described above, the weights and forward propagation defining the individual homolog layers 40 and 42 constitute at least part of a generator for synthesizing homologs passed from parents to offspring in a population consistent way. The homologs generated for each set of possible numerical values outputted from the middle layer (45 in FIG. 9) can then be used to simulate the allele ratios or reads obtained from a deletion, by ignoring one of the encoders 40 or 42, or another chromosomal abnormality. The value ranges selected for representing the output from the middle layer (45 in FIG. 9) may be selected, in order to generate realistic homologs, based on ranges of values close to the values that pass through the output of layer 39 in FIG. 9 when running validation or test data through the larger network starting from (37 in FIG. 9).

In some embodiments implement a GAN (e.g. as described above), after the GAN has been trained using population array or sequencing data for the microdeletion genomic region at hand, the homologs generated by the generative network of the GAN can be used to simulate the allele ratios or reads obtained from a deletion, by creating unphased genotypes using only a single homolog, or another chromosomal abnormality. The homologs can be used as synthetic data and can be used to augment and replace part of the training data as explained in connection with FIG. 8, and thereby enable the neural networks described above to detect related chromosomal abnormalities including microdeletions causing serious conditions in a fetus or embryo.

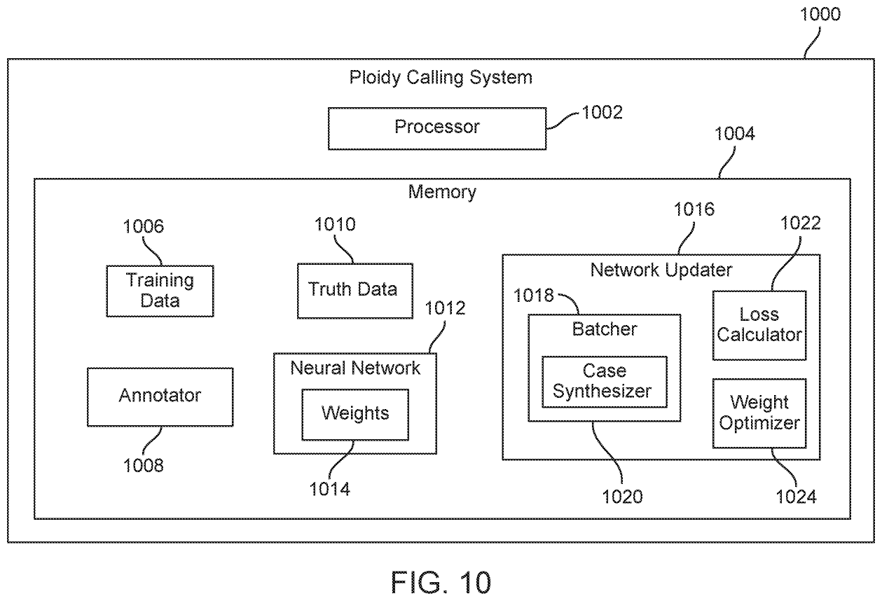

Referring now to FIG. 10, FIG. 10 is a block diagram showing an embodiment of an ploidy calling system 1000. The ploidy calling system 1000 can include one or more processors 1002, and a memory 1004. The one or more processors 1002 may include one or more microprocessors, application-specific integrated circuits (ASIC), a field-programmable gate arrays (FPGA), etc., or combinations thereof. The memory 1004 may include, but is not limited to, electronic, magnetic, or any other storage or transmission device capable of providing processor with program instructions. The memory may include magnetic disk, memory chip, read-only memory (ROM), random-access memory (RAM), Electrically Erasable Programmable Read-Only Memory (EEPROM), erasable programmable read only memory (EPROM), flash memory, or any other suitable memory from which processor can read instructions. The memory 1004 may include components, subsystems, modules, scripts, applications, or one or more sets of processor-executable instructions for implementing error analysis processes, including any processes described herein. For example, the memory 1004 may include training data 1006, an annotator 1008, a neural network 1012, truth data 1010, and a network updater 1016.

The training data 1006 may include genotyping or sequencing data for a genomic or plasma sample. The training data 1006 may be generated using, for example, a Cyto12b array or a targeted single nucleotide polymorphism (SNP) pool using Next Generation Sequencing (NGS). The Cyto12b array can have, for example, approximately 300 thousand (written here as ˜300 k) SNP targets across all chromosomes, and various NGS pools may, for example, have a smaller set of targeted SNPs ranging from hundreds of genomic positions to tens or hundreds of thousands of SNPs. The samples used to generate the training data 1006 may include, for example, one or more cells from an embryo, as well as optional genomic samples from parents of the embryo. In some embodiments, the samples may include a plasma sample from a pregnant mother (e.g. obtained by a non-invasive, with respect to the fetus, liquid biopsy). The training data 1006 may include numerical array data for each of the samples analyzed, which can include 2 or more numerical arrays of positive numbers per sample, where the length of each numerical array is equal to the number of genomic positions identified by the sequencing target pool or array and the individual entries in the numerical arrays.

The annotator 1008 may include components, subsystems, modules, scripts, applications, or one or more sets of processor-executable instructions for generating truth data using the training data. The annotator 1008 may apply empirical and first principal algorithms to the training data to annotate the training data (e.g. to classify the training data), to generate truth data 1010. The truth data 1010 can be used as reference data, and may be assumed to indicate, for example, an accurate classification of an analyzed sample. The truth data 1010 may include a classification and a likelihood of each chromosome identified from the embryos or fetus as being in a euploid state, or one of a number of ploidy states. In some embodiments, the annotator 1008 is used in conjunction with manual annotation to generate the truth data 1010. In some embodiments, the annotator 1008 may be omitted, and the truth data 1010 is generated or supplied in some other manner (e.g. via manual annotation).

The neural network 1012 may include components, subsystems, modules, scripts, applications, or one or more sets of processor-executable instructions for determining, for a test sample or during training, a ploidy state (e.g. a designation of euploidy or aneuploidy, or a designation of one or more specific aneuploidies) for a target genetic region by propagating genetic sequencing data or genetic array data (which may be pre-processed) through the neural network 1012. The neural network 1012 may output classification information that indicates the ploidy state. The neural network 1012 may include one or more layers. For example, the neural network 1012 may include multiple convolutional, activation and pooling layers (e.g. that reduce a size of an input vector, and extract relevant features in the form of additional channels). The neural network 1012 may include one or more series. The series may be chained or linked together. The series may extend to one or more series of fully connected layers, with dropout and other regularization techniques optionally embedded. The fully connected layers may have hundreds or thousands of nodes resulting in millions of weights 1014 between the nodes. The fully connected layers may be concatenated together to lead to a final layer. The neural network 1012 may include a final logits layer of size N by k where k is the number of classes in the classification desired (e.g. k=2 representing two classes: euploidy state and aneuploidy state). The final output of the neural network 1012 can, in some embodiments, be a single variable intended to indicate a statistical quantity such as the fetal fraction in the mother's plasma when such quantities are available in the truth set. The neural network 1012 may implement an “elu” activation function or a “ReLu” activation function. The neural network 1012 may include any of the features, structures, and may provide for any of the advantages, described herein, to output ploidy state information, and/or to call ploidy states.

The network updater 1016 may include components, subsystems, modules, scripts, applications, or one or more sets of processor-executable instructions for updating, optimizing, or modifying the neural network 1012. For example, the network updater 1016 may include a batcher 1018, a case synthesizer 1020, a loss calculator 1022, and a weight optimizer 1024. The network updater 1016 may be configured to modify the weights 1014 of the neural network 1012 to optimize the neural network 1012. For example, the network updater 1016 may feed batches of the training data 1006 through the neural network 1012 (each batch including one or more examples, or cases), and may optimize the neural network 1012 base on an output of such a process.

The batcher 1018 may include components, subsystems, modules, scripts, applications, or one or more sets of processor-executable instructions for determining batches of training data 1006 to pass through, or propagate through the neural network 1012. The batches may include a predetermined number of cases, or examples, of training data, each case corresponding to a respective genetic segment of the plurality of genetic segments and including data indicating an allele frequency for one or more positions of the respective genetic segment. The cases included in the batch may be randomly determined.

The batcher 1018 may include a case synthesizer 1020 configured to generate a synthetic case. For example, the batcher 1018 selects two cases from the training data 1006. This can be done randomly, and one of the cases (e.g. the second case) is picked from the training data 1006 so that it is guaranteed, by the truth data 1010, to have a whole chromosome or regional aneuploidy. For example, the case synthesizer 1020 can determine that the second case has a whole chromosome or regional aneuploidy, and can select the second case based on that determination. The case synthesizer 1020 selects (e.g. randomly) a segment, which may be of some minimum length, within the aneuploidy region of the second case and replaces the corresponding sequencing or array data from the first case by the data from the second case. The data replaced from the first case by data from the second case may correspond to the genomic positions from the aneuploidy segment selected from the second case. The case synthesizer 1020 may selectively (e.g. randomly or based on other criteria) pass the first case unchanged through the system so that during training the network may also be trained using unaltered examples. The case synthesizer 1020 may modify the truth data 1010 so that the inserted segment is counted as an aneuploidy segment in the modified first case when the case is submitted as part of a larger batch containing a mixture of synthetic and unaltered examples to the neural network during the training phase of the network. During the selection process, the batcher 1018 selects cases so that the sequencing or array data statistics found in the truth set or otherwise computed for the two examples is similar within a set range. In case of plasma from a pregnant mother this can include the two cases selected for producing the synthetic sequencing or array data possibly having a similar fetal fraction statistics. During training this procedure is repeated again during each epoch or cycle.

The loss calculator 1022 may be configured to determine, using a loss function or loss formula, one or more loss values based on the truth data 1010 and based on the output of the neural network 1012. For example, the loss formula includes a cross-entropy formula. The loss calculator 1022 may calculate a loss for a batch as a whole—for example, as the average or sum of the individual losses for each case included in the batch.

The weight optimizer 1024 is configured to optimize the weights 1014 and/or otherwise modify the neural network 1012 based on, for example, the loss values determined by the loss calculator 1022. The weight optimizer 1024 can modify the weights 1014 using, for example, a modified form of a stochastic gradient descent optimization, or another appropriate optimization process. In some embodiments, the weight optimizer 1024 uses a stochastic gradient descent-like algorithm with momentum (e.g. the Adam algorithm described herein, and sets the learning rate to about 0.0001. In some embodiments, the weight optimizer 1024 uses mini-batch gradient descent and momentum type optimization.

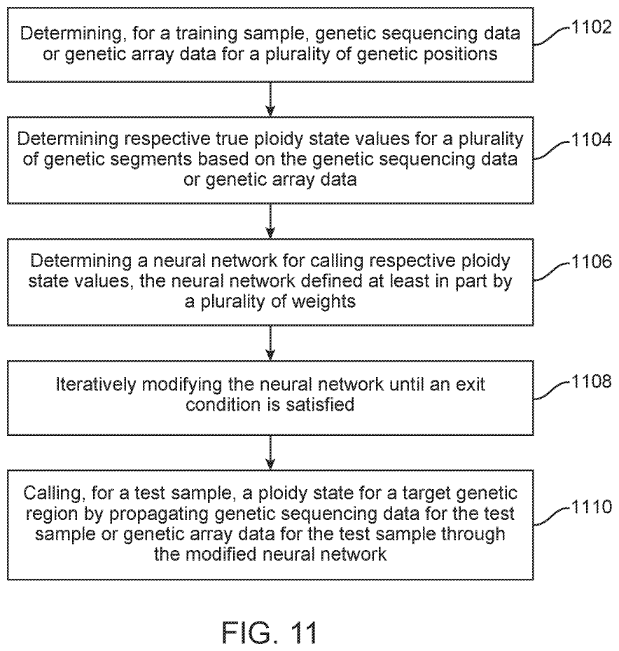

Referring now to FIG. 11, FIG. 11 is a flowchart showing an example method of calling a ploidy state for a target genetic region. The method includes processes 1102 through 1110. As a brief summary, in process 1102, the ploidy calling system 1000 determines, for a training sample, genetic sequencing data or genetic array data for a plurality of genetic positions. In process 1104, the ploidy calling system 1000 determines respective true ploidy state values for a plurality of genetic segments based on the genetic sequencing data or genetic array data. In process 1106, the ploidy calling system 1000 determines a neural network for calling respective ploidy state values, the neural network defined at least in part by a plurality of weights. In process 1108, the ploidy calling system 1000 iteratively modifying the neural network until an exit condition is satisfied. In process 1110, the ploidy calling system 1000 calls, for a test sample, a ploidy state for a target genetic region by propagating genetic sequencing data for the test sample or genetic array data for the test sample through the modified neural network.

In more detail, in process 1102, the ploidy calling system 1000 determines, for a training sample, genetic sequencing data or genetic array data for a plurality of genetic positions. The genetic sequencing data or genetic array data may include a Cyto12b array or a targeted single nucleotide polymorphism (SNP) pool using Next Generation Sequencing (NGS). The genetic sequencing data may include a number of reads or read counts of one or more targets. The Cyto12b array can have, for example, approximately 300 thousand (written here as ˜300 k) SNP targets across all chromosomes, and various NGS pools may, for example, have a smaller set of targeted SNPs ranging from hundreds of genomic positions to tens or hundreds of thousands of SNPs. The training sample used to generate the training data 1006 may include, for example, one or more cells from an embryo, as well as optional genomic samples from parents of the embryo. In some embodiments, the training sample may include a plasma sample from a pregnant mother (e.g. obtained by a non-invasive, with respect to the fetus, liquid biopsy).

In process 1104, the ploidy calling system 1000 determines respective true ploidy state values for a plurality of genetic segments based on the genetic sequencing data or genetic array data using the annotator 1008, which may apply empirical and first principal algorithms to the training data to annotate the training data (e.g. to classify the training data), to generate truth data 1010. The truth data 1010 can be used as reference data, and may be assumed to indicate, for example, an accurate classification of an analyzed sample. The truth data 1010 may include a classification and a likelihood of each chromosome identified from the embryos or fetus as being in a euploid state, or one of a number of aneuploidy states. In some embodiments, the annotator 1008 is used in conjunction with manual annotation to generate the truth data 1010. In some embodiments, the annotator 1008 may be omitted, and the truth data 1010 determined in some other manner such as via manual annotation, or by referencing an external database.

In process 1106, the ploidy calling system 1000 determines a neural network (e.g. the neural network 1012) for calling respective ploidy state values, the neural network defined at least in part by a plurality of weights. The neural network 1012 may output classification information that indicates the ploidy state. The neural network 1012 may include one or more layers. For example, the neural network 1012 may include multiple convolutional, activation and pooling layers (e.g. that reduce a size of an input vector, and extract relevant features in the form of additional channels). The neural network 1012 may include one or more series. The neural network 1012 may include a final logits layer of size N by k where k is the number of classes in the classification desired (e.g. k=2 representing two classes: euploidy state and aneuploidy state). The final output of the neural network 1012 can, in some embodiments, be a single variable intended to indicate a statistical quantity such as the fetal fraction in the mother's plasma when such quantities are available in the truth set. The neural network 1012 may implement an “elu” activation function or a “ReLu” activation function.

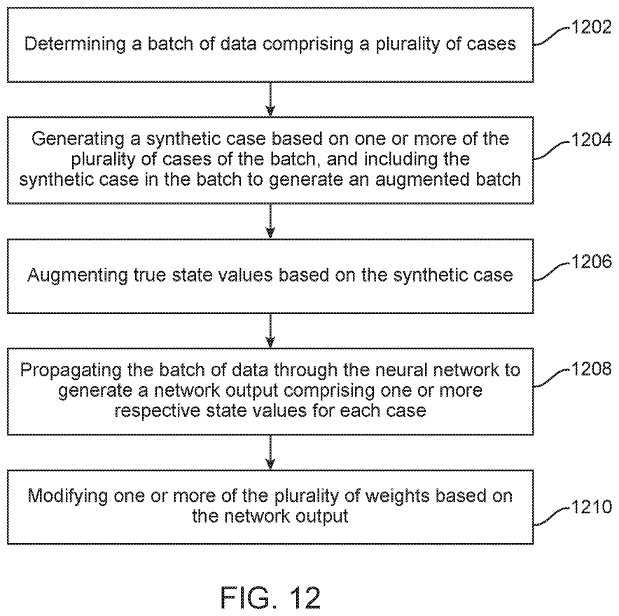

In process 1108, the ploidy calling system 1000 iteratively modifies (e.g. using the network updater 1016) the neural network until an exit condition is satisfied. The network updater 1016 may be configured to modify the weights 1014 of the neural network 1012 to optimize the neural network 1012. For example, the network updater 1016 may feed batches of the training data 1006 through the neural network 1012 (each batch including one or more examples, or cases), and may optimize the neural network 1012 base on an output of such a process (e.g. by minimizing a loss function). An example implementation of iteratively modifying the neural network is shown in FIG. 12.

In process 1110, the ploidy calling system 1000 calls, for a test sample, a ploidy state for a target genetic region by propagating genetic sequencing data for the test sample or genetic array data for the test sample through the modified neural network. In some embodiments, a network output is a classification vector such as (x,y) with x and y numerical non-negative values that sum to 1 and where x>>y indicates a euploid classification and y>>x indicates an aneuploid classification of the embryo. For example, if the x value is greater than the y value by a predetermined amount (which may, in some embodiments, be zero, or a negative amount), the system may classify the sample as euploid, and if the y value is greater than the x value by a predetermined amount (which may, in some embodiments, be zero, or a negative amount), the system may classify the sample as exhibiting aneuploidy.

Referring now to FIG. 12, FIG. 12 is a flowchart showing an example method of modifying a neural network. The example method may be used iteratively to optimize a neural network. The method includes processes 1202 through 1210. As a brief summary, in process 1202, the ploidy calling system 1000 determines a batch of data comprising a plurality of cases. In process 1204, the ploidy calling system 1000 generates a synthetic case based on one or more of the plurality of cases of the batch, and includes the synthetic case in the batch to generate an augmented batch. In process 1206, the ploidy calling system 1000 augments the true state values based on the synthetic case. In process 1208, the ploidy calling system 1000 propagates the batch of data through the neural network to generate a network output comprising one or more respective state values for each case. In process 1210, the ploidy calling system 1000 modifies one or more of the plurality of weights based on the network output.

In more detail, in process 1202, the ploidy calling system 1000 determines (e.g. using the batcher 1018) a batch of data comprising a plurality of cases. The batcher 1018 may include components, subsystems, modules, scripts, applications, or one or more sets of processor-executable instructions for determining batches of training data to pass through, or propagate through the neural network. The batches may include a predetermined number of cases, or examples, of training data, each case corresponding to a respective genetic segment of the plurality of genetic segments and including data indicating an allele frequency for one or more positions of the respective genetic segment. The cases included in the batch may be randomly determined.

In process 1204, the ploidy calling system 1000 generates (e.g. using a case synthesizer 1020) a synthetic case based on one or more of the plurality of cases of the batch, and includes the synthetic case in the batch to generate an augmented batch. For example, the batcher 1018 selects two cases from the training data 1006. This can be done randomly, and one of the cases (e.g. the second case) is picked from the training data so that it is guaranteed, by the truth data, to have a whole chromosome or regional aneuploidy. For example, the case synthesizer 1020 can determine that the second case has a whole chromosome or regional aneuploidy, and can select the second case based on that determination. The case synthesizer 1020 selects (e.g. randomly) a segment, which may be of some minimum length, within the aneuploidy region of the second case and replaces the corresponding sequencing or array data from the first case by the data from the second case. The data replaced from the first case by data from the second case may correspond to the genomic positions from the aneuploidy segment selected from the second case. The case synthesizer 1020 may selectively (e.g. randomly or based on other criteria) pass the first case unchanged through the system so that during training the network may also be trained using unaltered examples. During the selection process, the batcher 1018 selects cases so that the sequencing or array data statistics found in the truth set or otherwise computed for the two examples is similar within a set range. In case of plasma from a pregnant mother this can include the two cases selected for producing the synthetic sequencing or array data possibly having a similar fetal fraction statistics. During training this procedure is repeated again during each epoch or cycle.

In process 1206, the ploidy calling system 1000 augments the true state values based on the synthetic case. The case synthesizer 1020 may modify the truth data 1010 so that the inserted segment is counted as an aneuploidy segment in the modified first case when the case is submitted as part of a larger batch containing a mixture of synthetic and unaltered examples to the neural network during the training phase of the network.

In process 1208, the ploidy calling system 1000 propagates the batch of data through the neural network to generate a network output comprising one or more respective state values for each case. In process 1210, the ploidy calling system 1000 modifies one or more of the plurality of weights based on the network output. This may be implemented, for example, using the weight optimizer 1024 and based on, for example, the loss values determined by the loss calculator 1022. The weight optimizer 1024 can modify the weights of the neural network using, for example, a modified form of a stochastic gradient descent optimization, or another appropriate optimization process. In some embodiments, the weight optimizer 1024 uses a stochastic gradient descent-like algorithm with momentum (e.g. the Adam algorithm described herein), and sets the learning rate to about 0.0001. In some embodiments, the weight optimizer 1024 uses mini-batch gradient descent and momentum type optimization. Thus, the ploidy calling system 1000 may train the neural network.

### Sample Preparation

In some embodiments, the system and methods described herein may be used to call a ploidy state for a biological sample. The biological sample may be fetal, maternal, or paternal. The biological sample may be selected from blood, serum, plasma, urine, and a biopsy sample. In some embodiments, at least 10, or at least 20, or at least 50, or at least 100, or at least 200, or at least 500, or at least 1,000 SNV loci are amplified from the isolated cell-free DNA. In some embodiments, the amplification products are sequenced with a depth of read of at least 200, or at least 500, or at least 1,000, or at least 2,000, or at least 5,000, or at least 10,000, or at least 20,000, or at least 50,000, or at least 100,000. Preparation or processing of the sample may include isolating cell-free DNA from a biological sample of a subject, amplifying from the isolated cell-free DNA a plurality of single-nucleotide variant (SNV) loci that comprise a plurality of target bases, and sequencing the amplification products to obtain genetic sequencing data. Some embodiments include collecting and analyzing a plurality of biological samples from the patient longitudinally.

### Methods for Detecting Cancer

In a further aspect, the present disclosure provides a method for classifying a sample as cancerous, comprising: isolating cell-free DNA from a biological sample of a subject; amplifying from the isolated cell-free DNA a plurality of single-nucleotide variant (SNV) loci or segements that comprise a plurality of target bases, wherein the SNV loci or segments are known to be associated with cancer; sequencing the amplification products; and using one or more processes described herein (e.g., making use of a neural network trained in a manner described herein, which may make use of labelled, augmented, and/or synthesized training data) to classifying the sample as cancerous. In some embodiments, the plurality of single nucleotide variance loci are selected from SNV loci identified in the TCGA and COSMIC data sets for cancer.

Some embodiments include performing a multiplex amplification reaction to amplify from the isolated cell-free DNA for a plurality of single-nucleotide variant (SNV) loci that comprise a plurality of target bases, wherein the SNV loci are patient-specific SNV loci associated with the cancer for which the subject has received treatment; and sequencing the amplification products to obtain sequence reads of the plurality of target bases. In some embodiments, the multiplex amplification reaction amplifies at least 4, or at least 8, or at least 16, or at least 32, or at least 64, or at least 128 patient-specific SNV loci associated with the cancer for which the subject has received treatment.

The terms “cancer” and “cancerous” refer to or describe the physiological condition in animals that is typically characterized by unregulated cell growth. A “tumor” comprises one or more cancerous cells. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue, such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments, the cancer comprises an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sezary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenstrom macroglobulinemia; or Wilm's tumor.

In certain examples, the methods includes identifying a confidence value for each allele determination at each of the set of single nucleotide variance loci, which can be based at least in part on a depth of read for the loci. The confidence limit can be set at least 75%, 80%, 85%, 90%, 95%, 96%, 96%, 98%, or 99%. The confidence limit can be set at different levels for different types of mutations

In any of the methods for detecting SNVs herein that include a ctDNA SNV amplification/sequencing workflow, improved amplification parameters for multiplex PCR can be employed. For example, wherein the amplification reaction is a PCR reaction and the annealing temperature is between 1, 2, 3, 4, 5, 6, 7, 8, 9, or 10° C. greater than the melting temperature on the low end of the range, and 2, 3, 4, 5, 6, 7, 8, 9, 10, 11, 12, 13, 14 or 15° on the high end the range for at least 10, 20, 25, 30, 40, 50, 06, 70, 75, 80, 90, 95 or 100% the primers of the set of primers.

In certain embodiments, wherein the amplification reaction is a PCR reaction the length of the annealing step in the PCR reaction is between 10, 15, 20, 30, 45, and 60 minutes on the low end of the range, and 15, 20, 30, 45, 60, 120, 180, or 240 minutes on the high end of the range. In certain embodiments, the primer concentration in the amplification, such as the PCR reaction is between 1 and 10 nM. Furthermore, in exemplary embodiments, the primers in the set of primers, are designed to minimize primer dimer formation.

Accordingly, in an example of any of the methods herein that include an amplification step, the amplification reaction is a PCR reaction, the annealing temperature is between 1 and 10° C. greater than the melting temperature of at least 90% of the primers of the set of primers, the length of the annealing step in the PCR reaction is between 15 and 60 minutes, the primer concentration in the amplification reaction is between 1 and 10 nM, and the primers in the set of primers, are designed to minimize primer dimer formation. In a further aspect of this example, the multiplex amplification reaction is performed under limiting primer conditions.

A sample analyzed in methods of the present invention, in certain illustrative embodiments, is a blood sample, or a fraction thereof. Methods provided herein, in certain embodiments, are specially adapted for amplifying DNA fragments, especially tumor DNA fragments that are found in circulating tumor DNA (ctDNA). Such fragments are typically about 160 nucleotides in length.

It is known in the art that cell-free nucleic acid (e.g. cfDNA), can be released into the circulation via various forms of cell death such as apoptosis, necrosis, autophagy and necroptosis. The cfDNA, is fragmented and the size distribution of the fragments varies from 150-350 bp to >10000 bp. (see Kalnina et al. World J Gastroenterol. 2015 Nov. 7; 21(41): 11636-11653). For example the size distributions of plasma DNA fragments in hepatocellular carcinoma (HCC) patients spanned a range of 100-220 bp in length with a peak in count frequency at about 166 bp and the highest tumor DNA concentration in fragments of 150-180 bp in length (see: Jiang et al. Proc Nati Acad Sci USA 112:E1317-E1325).

In an illustrative embodiment the circulating tumor DNA (ctDNA) is isolated from blood using EDTA-2Na tube after removal of cellular debris and platelets by centrifugation. The plasma samples can be stored at −80° C. until the DNA is extracted using, for example, QIAamp DNA Mini Kit (Qiagen, Hilden, Germany), (e.g. Hamakawa et al., Br J Cancer. 2015; 112:352-356). Hamakava et al. reported median concentration of extracted cell free DNA of all samples 43.1 ng per ml plasma (range 9.5-1338 ng ml) and a mutant fraction range of 0.001-77.8%, with a median of 0.90%.

Methods of the present description, in certain embodiments, include a step of generating and amplifying a nucleic acid library from the sample (i.e. library preparation). The nucleic acids from the sample during the library preparation step can have ligation adapters, often referred to as library tags or ligation adaptor tags (LTs), appended, where the ligation adapters contain a universal priming sequence, followed by a universal amplification. In an embodiment, this may be done using a standard protocol designed to create sequencing libraries after fragmentation. In an embodiment, the DNA sample can be blunt ended, and then an A can be added at the 3′ end. A Y-adaptor with a T-overhang can be added and ligated. In some embodiments, other sticky ends can be used other than an A or T overhang. In some embodiments, other adaptors can be added, for example looped ligation adaptors. In some embodiments, the adaptors may have tag designed for PCR amplification.

A number of the embodiments provided herein, include detecting the SNVs in a ctDNA sample. Such methods in illustrative embodiments, include an amplification step and a sequencing step (sometimes referred to herein as a “ctDNA SNV amplification/sequencing workflow). In an illustrative example, a ctDNA amplification/sequencing workflow can include generating a set of amplicons by performing a multiplex amplification reaction on nucleic acids isolated from a sample of blood or a fraction thereof from an individual, such as an individual suspected of having cancer wherein each amplicon of the set of amplicons spans at least one single nucleotide variant loci of a set of single nucleotide variant loci, such as an SNV loci known to be associated with cancer; and determining the sequence of at least a segment of at each amplicon of the set of amplicons, wherein the segment comprises a single nucleotide variant loci. In this way, this exemplary method determines the single nucleotide variants present in the sample.

Exemplary ctDNA SNV amplification/sequencing workflows in more detail can include forming an amplification reaction mixture by combining a polymerase, nucleotide triphosphates, nucleic acid fragments from a nucleic acid library generated from the sample, and a set of primers that each binds an effective distance from a single nucleotide variant loci, or a set of primer pairs that each span an effective region that includes a single nucleotide variant loci. The single nucleotide variant loci, in exemplary embodiments, is one known to be associated with cancer. Then, subjecting the amplification reaction mixture to amplification conditions to generate a set of amplicons comprising at least one single nucleotide variant loci of a set of single nucleotide variant loci, preferably known to be associated with cancer; and determining the sequence of at least a segment of each amplicon of the set of amplicons, wherein the segment comprises a single nucleotide variant loci.

The effective distance of binding of the primers can be within 1, 2, 3, 4, 5, 6, 7, 8, 9, 10, 11, 12, 13, 14, 15, 20, 25, 30, 35, 40, 45, 50, 75, 100, 125, or 150 base pairs of a SNV loci. The effective range that a pair of primers spans typically includes an SNV and is typically 160 base pairs or less, and can be 150, 140, 130, 125, 100, 75, 50 or 25 base pairs or less. In other embodiments, the effective range that a pair of primers spans is 20, 25, 30, 40, 50, 60, 70, 75, 100, 110, 120, 125, 130, 140, or 150 nucleotides from an SNV loci on the low end of the range, and 25, 30, 40, 50, 60, 70, 75, 100, 110, 120, 125, 130, 140, or 150, 160, 170, 175, or 200 on the high end of the range.

Primer tails can improve the detection of fragmented DNA from universally tagged libraries. If the library tag and the primer-tails contain a homologous sequence, hybridization can be improved (for example, melting temperature (Tm) is lowered) and primers can be extended if only a portion of the primer target sequence is in the sample DNA fragment. In some embodiments, 13 or more target specific base pairs may be used. In some embodiments, 10 to 12 target specific base pairs may be used. In some embodiments, 8 to 9 target specific base pairs may be used. In some embodiments, 6 to 7 target specific base pairs may be used.

In one embodiment, libraries are generated from the samples above by ligating adaptors to the ends of DNA fragments in the samples, or to the ends of DNA fragments generated from DNA isolated from the samples. The fragments can then be amplified using PCR, for example, according to the following exemplary protocol: 95° C., 2 min; 15×[95° C., 20 sec, 55° C., 20 sec, 68° C., 20 sec], 68° C. 2 min, 4° C. hold.

Many kits and methods are known in the art for generation of libraries of nucleic acids that include universal primer binding sites for subsequent amplification, for example clonal amplification, and for subsequence sequencing. To help facilitate ligation of adapters library preparation and amplification can include end repair and adenylation (i.e. A-tailing). Kits especially adapted for preparing libraries from small nucleic acid fragments, especially circulating free DNA, can be useful for practicing methods provided herein. For example, the NEXTflex Cell Free kits available from Bioo Scientific ( ) or the Natera Library Prep Kit (available from Natera, Inc. San Carlos, Calif.). However, such kits would typically be modified to include adaptors that are customized for the amplification and sequencing steps of the methods provided herein. Adaptor ligation can be performed using commercially available kits such as the ligation kit found in the AGILENT SURESELECT kit (Agilent, Calif.).

Target regions of the nucleic acid library generated from DNA isolated from the sample, especially a circulating free DNA sample for the methods of the present invention, are then amplified. For this amplification, a series of primers or primer pairs, which can include between 5, 10, 15, 20, 25, 50, 100, 125, 150, 250, 500, 1000, 2500, 5000, 10,000, 20,000, 25,000, or 50,000 on the low end of the range and 15, 20, 25, 50, 100, 125, 150, 250, 500, 1000, 2500, 5000, 10,000, 20,000, 25,000, 50,000, 60,000, 75,000, or 100,000 primers on the upper end of the range, that each bind to one of a series of primer binding sites.

Primer designs can be generated with Primer3 (Untergrasser A, Cutcutache I, Koressaar T, Ye J, Faircloth B C, Remm M, Rozen S G (2012) “Primer3—new capabilities and interfaces.” Nucleic Acids Research 40(15):e115 and Koressaar T, Remm M (2007) “Enhancements and modifications of primer design program Primer3.” Bioinformatics 23(10):1289-91) source code available at primer3.sourceforge.net). Primer specificity can be evaluated by BLAST and added to existing primer design pipeline criteria:

Primer specificities can be determined using the BLASTn program from the ncbi-blast-2.2.29+ package. The task option “blastn-short” can be used to map the primers against hg19 human genome. Primer designs can be determined as “specific” if the primer has less than 100 hits to the genome and the top hit is the target complementary primer binding region of the genome and is at least two scores higher than other hits (score is defined by BLASTn program). This can be done in order to have a unique hit to the genome and to not have many other hits throughout the genome.

The final selected primers can be visualized in IGV (James T. Robinson, Helga Thorvaldsdottir, Wendy Winckler, Mitchell Guttman, Eric S. Lander, Gad Getz, Jill P. Mesirov. Integrative Genomics Viewer. Nature Biotechnology 29, 24-26 (2011)) and UCSC browser (Kent W J, Sugnet C W, Furey T S, Roskin K M, Pringle T H, Zahler A M, Haussler D. The human genome browser at UCSC. Genome Res. 2002 June; 12(6):996-1006) using bed files and coverage maps for validation.

Methods described herein, in certain embodiments, include forming an amplification reaction mixture. The reaction mixture typically is formed by combining a polymerase, nucleotide triphosphates, nucleic acid fragments from a nucleic acid library generated from the sample, a set of forward and reverse primers specific for target regions that contain SNVs. The reaction mixtures provided herein, themselves forming in illustrative embodiments, a separate aspect of the invention.

An amplification reaction mixture useful for the present invention includes components known in the art for nucleic acid amplification, especially for PCR amplification. For example, the reaction mixture typically includes nucleotide triphosphates, a polymerase, and magnesium. Polymerases that are useful for the present invention can include any polymerase that can be used in an amplification reaction especially those that are useful in PCR reactions. In certain embodiments, hot start Taq polymerases are especially useful. Amplification reaction mixtures useful for practicing the methods provided herein, such as AmpliTaq Gold master mix (Life Technologies, Carlsbad, Calif.), are available commercially.

Amplification (e.g. temperature cycling) conditions for PCR are well known in the art. The methods provided herein can include any PCR cycling conditions that result in amplification of target nucleic acids such as target nucleic acids from a library. Non-limiting exemplary cycling conditions are provided in the Examples section herein.

There are many workflows that are possible when conducting PCR; some workflows typical to the methods disclosed herein are provided herein. The steps outlined herein are not meant to exclude other possible steps nor does it imply that any of the steps described herein are required for the method to work properly. A large number of parameter variations or other modifications are known in the literature, and may be made without affecting the essence of the invention.

In certain embodiments of the method provided herein, at least a portion and in illustrative examples the entire sequence of an amplicon, such as an outer primer target amplicon, is determined. Methods for determining the sequence of an amplicon are known in the art. Any of the sequencing methods known in the art, e.g. Sanger sequencing, can be used for such sequence determination. In illustrative embodiments high throughput next-generation sequencing techniques (also referred to herein as massively parallel sequencing techniques) such as, but not limited to, those employed in MYSEQ (ILLUMINA), HISEQ (ILLUMINA), ION TORRENT (LIFE TECHNOLOGIES), GENOME ANALYZER ILX (ILLUMINA), GS FLEX+ (ROCHE 454), can be used for sequencing the amplicons produced by the methods provided herein.

High throughput genetic sequencers are amenable to the use of barcoding (i.e., sample tagging with distinctive nucleic acid sequences) so as to identify specific samples from individuals thereby permitting the simultaneous analysis of multiple samples in a single run of the DNA sequencer. The number of times a given region of the genome in a library preparation (or other nucleic preparation of interest) is sequenced (number of reads) will be proportional to the number of copies of that sequence in the genome of interest (or expression level in the case of cDNA containing preparations). Biases in amplification efficiency can be taken into account in such quantitative determination.

Target Genes. Target genes of the present invention in exemplary embodiments, are cancer-related genes, and in many illustrative embodiments, cancer-related genes. A cancer-related gene refers to a gene associated with an altered risk for a cancer or an altered prognosis for a cancer. Exemplary cancer-related genes that promote cancer include oncogenes; genes that enhance cell proliferation, invasion, or metastasis; genes that inhibit apoptosis; and pro-angiogenesis genes. Cancer-related genes that inhibit cancer include, but are not limited to, tumor suppressor genes; genes that inhibit cell proliferation, invasion, or metastasis; genes that promote apoptosis; and anti-angiogenesis genes.

An embodiment of a method for calling a ploidy state begins with the selection of the region of the gene or loci that becomes the target. The region with known mutations is used to develop primers for mPCR-NGS to amplify and detect the mutation.

Methods provided herein can be used to detect virtually any type of mutation, including mutations known to be associated with cancer and most particularly the methods provided herein are directed to mutations, especially SNVs, associated with cancer. Exemplary SNVs can be in one or more of the following genes: EGFR, FGFR1, FGFR2, ALK, MET, ROS1, NTRK1, RET, HER2, DDR2, PDGFRA, KRAS, NF1, BRAF, PIK3CA, MEK1, NOTCH1, MLL2, EZH2, TET2, DNMT3A, SOX2, MYC, KEAP1, CDKN2A, NRG1, TP53, LKB1, and PTEN, which have been identified in various lung cancer samples as being mutated, having increased copy numbers, or being fused to other genes and combinations thereof (Non-small-cell lung cancers: a heterogeneous set of diseases. Chen et al. Nat. Rev. Cancer. 2014 Aug. 14(8):535-551). In another example, the list of genes are those listed above, where SNVs have been reported, such as in the cited Chen et al. reference.

Other exemplary polymorphisms or mutations are in one or more of the following genes: TP53, PTEN, PIK3CA, APC, EGFR, NRAS, NF2, FBXW7, ERBBs, ATAD5, KRAS, BRAF, VEGF, EGFR, HER2, ALK, p53, BRCA, BRCA1, BRCA2, SETD2, LRP1B, PBRM, SPTA1, DNMT3A, ARID1A, GRIN2A, TRRAP, STAG2, EPHA3/5/7, POLE, SYNE1, C20orf80, CSMD1, CTNNB1, ERBB2. FBXW7, KIT, MUC4, ATM, CDH1, DDX11, DDX12, DSPP, EPPK1, FAM186A, GNAS, HRNR, KRTAP4-11, MAP2K4, MLL3, NRAS, RB1, SMAD4, TTN, ABCC9, ACVR1B, ADAM29, ADAMTS19, AGAP10, AKT1, AMBN, AMPD2, ANKRD30A, ANKRD40, APOBR, AR, BIRC6, BMP2, BRAT1, BTNL8, C12orf4, C1QTNF7, C20orf186, CAPRIN2, CBWD1, CCDC30, CCDC93, CD5L, CDC27, CDC42BPA, CDH9, CDKN2A, CHD8, CHEK2, CHRNA9, CIZ1, CLSPN, CNTN6, COL14A1, CREBBP, CROCC, CTSF, CYP1A2, DCLK1, DHDDS, DHX32, DKK2, DLEC1, DNAH14, DNAH5, DNAH9, DNASE1L3, DUSP16, DYNC2H1, ECT2, EFHB, RRN3P2, TRIM49B, TUBB8P5, EPHA7, ERBB3, ERCC6, FAM21A, FAM21C, FCGBP, FGFR2, FLG2, FLT1, FOLR2, FRYL, FSCB, GAB1, GABRA4, GABRP, GH2, GOLGA6L1, GPHB5, GPR32, GPX5, GTF3C3, HECW1, HIST1H3B, HLA-A, HRAS, HS3ST1, HS6ST1, HSPD1, IDH1, JAK2, KDM5B, KIAA0528, KRT15, KRT38, KRTAP21-1, KRTAP4-5, KRTAP4-7, KRTAP5-4, KRTAP5-5, LAMA4, LATS1, LMF1, LPAR4, LPPR4, LRRFIP1, LUM, LYST, MAP2K1, MARCH1, MARCO, MB21D2, MEGF10, MMP16, MORC1, MRE11A, MTMR3, MUC12, MUC17, MUC2, MUC20, NBPF10, NBPF20, NEK1, NFE2L2, NLRP4, NOTCH2, NRK, NUP93, OBSCN, OR11H1, OR2B11, OR2M4, OR4Q3, OR5D13, OR8I2, OXSM, PIK3R1, PPP2R5C, PRAME, PRF1, PRG4, PRPF19, PTH2, PTPRC, PTPRJ, RAC1, RAD50, RBM12, RGPD3, RGS22, ROR1, RP11-671M22.1, RP13-996F3.4, RP1L1, RSBN1L, RYR3, SAMD3, SCN3A, SEC31A, SF1, SF3B1, SLC25A2, SLC44A1, SLC4A11, SMAD2, SPTA1, ST6GAL2, STK11, SZT2, TAF1L, TAX1BP1, TBP, TGFBI, TIF1, TMEM14B, TMEM74, TPTE, TRAPPC8, TRPS1, TXNDC6, USP32, UTP20, VASN, VPS72, WASH3P, WWTR1, XPO1, ZFHX4, ZMIZ1, ZNF167, ZNF436, ZNF492, ZNF598, ZRSR2, ABL1, AKT2, AKT3, ARAF, ARFRP1, ARID2, ASXL1, ATR, ATRX, AURKA, AURKB, AXL, BAP1, BARD1, BCL2, BCL2L2, BCL6, BCOR, BCORL1, BLM, BRIP1, BTK, CARD11, CBFB, CBL, CCND1, CCND2, CCND3, CCNE1, CD79A, CD79B, CDC73, CDK12, CDK4, CDK6, CDK8, CDKN1B, CDKN2B, CDKN2C, CEBPA, CHEK1, CIC, CRKL, CRLF2, CSF1R, CTCF, CTNNA1, DAXX, DDR2, DOT1L, EMSY (C11orf30), EP300, EPHA3, EPHA5, EPHB1, ERBB4, ERG, ESR1, EZH2, FAM123B (WTX), FAM46C, FANCA, FANCC, FANCD2, FANCE, FANCF, FANCG, FANCL, FGF10, FGF14, FGF19, FGF23, FGF3, FGF4, FGF6, FGFR1, FGFR2, FGFR3, FGFR4, FLT3, FLT4, FOXL2, GATA1, GATA2, GATA3, GID4 (C17orf39), GNA11, GNA13, GNAQ, GNAS, GPR124, GSK3B, HGF, IDH1, IDH2, IGF1R, IKBKE, IKZF1, IL7R, INHBA, IRF4, IRS2, JAK1, JAK3, JUN, KAT6A (MYST3), KDM5A, KDM5C, KDM6A, KDR, KEAP1, KLHL6, MAP2K2, MAP2K4, MAP3K1, MCL1, MDM2, MDM4, MED12, MEF2B, MEN1, MET, MITF, MLH1, MLL, MLL2, MPL, MSH2, MSH6, MTOR, MUTYH, MYC, MYCL1, MYCN, MYD88, NF1, NFKBIA, NKX2-1, NOTCH1, NPM1, NRAS, NTRK1, NTRK2, NTRK3, PAK3, PALB2, PAX5, PBRM1, PDGFRA, PDGFRB, PDK1, PIK3CG, PIK3R2, PPP2R1A, PRDM1, PRKAR1A, PRKDC, PTCH1, PTPN11, RAD51, RAF1, RARA, RET, RICTOR, RNF43, RPTOR, RUNX1, SMARCA4, SMARCB1, SMO, SOCS1, SOX10, SOX2, SPEN, SPOP, SRC, STAT4, SUFU, TET2, TGFBR2, TNFAIP3, TNFRSF14, TOP1, TP53, TSC1, TSC2, TSHR, VHL, WISP3, WT1, ZNF217, ZNF703, and combinations thereof (Su et al., J Mol Diagn 2011, 13:74-84; DOI:10.1016/j.jmoldx.2010.11.010; and Abaan et al., “The Exomes of the NCI-60 Panel: A Genomic Resource for Cancer Biology and Systems Pharmacology”, Cancer Research, Jul. 15, 2013, which are each hereby incorporated by reference in its entirety). Exemplary polymorphisms or mutations can be in one or more of the following microRNAs: miR-15a, miR-16-1, miR-23a, miR-23b, miR-24-1, miR-24-2, miR-27a, miR-27b, miR-29b-2, miR-29c, miR-146, miR-155, miR-221, miR-222, and miR-223 (Calin et al. “A microRNA signature associated with prognosis and progression in chronic lymphocytic leukemia.” N Engl J Med 353:1793-801, 2005, which is hereby incorporated by reference in its entirety).

Amplification (e.g. PCR) Reaction Mixtures

Methods of the present description, in certain embodiments, include forming an amplification reaction mixture. The reaction mixture typically is formed by combining a polymerase, nucleotide triphosphates, nucleic acid fragments from a nucleic acid library generated from the sample, a series of forward target-specific outer primers and a first strand reverse outer universal primer. Another illustrative embodiment is a reaction mixture that includes forward target-specific inner primers instead of the forward target-specific outer primers and amplicons from a first PCR reaction using the outer primers, instead of nucleic acid fragments from the nucleic acid library. The reaction mixtures provided herein, themselves forming in illustrative embodiments, a separate aspect of the invention. In illustrative embodiments, the reaction mixtures are PCR reaction mixtures. PCR reaction mixtures typically include magnesium.

In some embodiments, the reaction mixture includes ethylenediaminetetraacetic acid (EDTA), magnesium, tetramethyl ammonium chloride (TMAC), or any combination thereof. In some embodiments, the concentration of TMAC is between 20 and 70 mM, inclusive. While not meant to be bound to any particular theory, it is believed that TMAC binds to DNA, stabilizes duplexes, increases primer specificity, and/or equalizes the melting temperatures of different primers. In some embodiments, TMAC increases the uniformity in the amount of amplified products for the different targets. In some embodiments, the concentration of magnesium (such as magnesium from magnesium chloride) is between 1 and 8 mM.

The large number of primers used for multiplex PCR of a large number of targets may chelate a lot of the magnesium (2 phosphates in the primers chelate 1 magnesium). For example, if enough primers are used such that the concentration of phosphate from the primers is ˜9 mM, then the primers may reduce the effective magnesium concentration by ˜4.5 mM. In some embodiments, EDTA is used to decrease the amount of magnesium available as a cofactor for the polymerase since high concentrations of magnesium can result in PCR errors, such as amplification of non-target loci. In some embodiments, the concentration of EDTA reduces the amount of available magnesium to between 1 and 5 mM (such as between 3 and 5 mM).

In some embodiments, the pH is between 7.5 and 8.5, such as between 7.5 and 8, 8 and 8.3, or 8.3 and 8.5, inclusive. In some embodiments, Tris is used at, for example, a concentration of between 10 and 100 mM, such as between 10 and 25 mM, 25 and 50 mM, 50 and 75 mM, or 25 and 75 mM, inclusive. In some embodiments, any of these concentrations of Tris are used at a pH between 7.5 and 8.5. In some embodiments, a combination of KCl and (NH₄)₂SO₄ is used, such as between 50 and 150 mM KCl and between 10 and 90 mM (NH₄)₂SO₄, inclusive. In some embodiments, the concentration of KCl is between 0 and 30 mM, between 50 and 100 mM, or between 100 and 150 mM, inclusive. In some embodiments, the concentration of (NH₄)₂SO₄ is between 10 and 50 mM, 50 and 90 mM, 10 and 20 mM, 20 and 40 mM, 40 and 60 mM, or 60 and 80 mM (NH₄)₂SO₄, inclusive. In some embodiments, the ammonium [NH₄⁺] concentration is between 0 and 160 mM, such as between 0 to 50, 50 to 100, or 100 to 160 mM, inclusive. In some embodiments, the sum of the potassium and ammonium concentration ([K⁺]+[NH₄⁺]) is between 0 and 160 mM, such as between 0 to 25, 25 to 50, 50 to 150, 50 to 75, 75 to 100, 100 to 125, or 125 to 160 mM, inclusive. An exemplary buffer with [K⁺]+[NH₄⁺]=120 mM is 20 mM KCl and 50 mM (NH₄)₂SO₄. In some embodiments, the buffer includes 25 to 75 mM Tris, pH 7.2 to 8, 0 to 50 mM KCl, 10 to 80 mM ammonium sulfate, and 3 to 6 mM magnesium, inclusive. In some embodiments, the buffer includes 25 to 75 mM Tris pH 7 to 8.5, 3 to 6 mM MgCl₂, 10 to 50 mM KCl, and 20 to 80 mM (NH₄)₂SO₄, inclusive. In some embodiments, 100 to 200 Units/mL of polymerase are used. In some embodiments, 100 mM KCl, 50 mM (NH₄)₂SO₄, 3 mM MgCl₂, 7.5 nM of each primer in the library, 50 mM TMAC, and 7 ul DNA template in a 20 ul final volume at pH 8.1 is used.

In some embodiments, a crowding agent is used, such as polyethylene glycol (PEG, such as PEG 8,000) or glycerol. In some embodiments, the amount of PEG (such as PEG 8,000) is between 0.1 to 20%, such as between 0.5 to 15%, 1 to 10%, 2 to 8%, or 4 to 8%, inclusive. In some embodiments, the amount of glycerol is between 0.1 to 20%, such as between 0.5 to 15%, 1 to 10%, 2 to 8%, or 4 to 8%, inclusive. In some embodiments, a crowding agent allows either a low polymerase concentration and/or a shorter annealing time to be used. In some embodiments, a crowding agent improves the uniformity of the DOR and/or reduces dropouts (undetected alleles).

In some embodiments, a polymerase with proof-reading activity, a polymerase without (or with negligible) proof-reading activity, or a mixture of a polymerase with proof-reading activity and a polymerase without (or with negligible) proof-reading activity is used. In some embodiments, a hot start polymerase, a non-hot start polymerase, or a mixture of a hot start polymerase and a non-hot start polymerase is used. In some embodiments, a HotStarTaq DNA polymerase is used (see, for example, QIAGEN catalog No. 203203). In some embodiments, AmpliTaq Gold® DNA Polymerase is used. In some embodiments a PrimeSTAR GXL DNA polymerase, a high fidelity polymerase that provides efficient PCR amplification when there is excess template in the reaction mixture, and when amplifying long products, is used (Takara Clontech, Mountain View, Calif.). In some embodiments, KAPA Taq DNA Polymerase or KAPA Taq HotStart DNA Polymerase is used; they are based on the single-subunit, wild-type Taq DNA polymerase of the thermophilic bacterium Thermus aquaticus. KAPA Taq and KAPA Taq HotStart DNA Polymerase have 5′-3′ polymerase and 5′-3′ exonuclease activities, but no 3′ to 5′ exonuclease (proofreading) activity (see, for example, KAPA BIOSYSTEMS catalog No. BK1000). In some embodiments, Pfu DNA polymerase is used; it is a highly thermostable DNA polymerase from the hyperthermophilic archaeum Pyrococcus furiosus. The enzyme catalyzes the template-dependent polymerization of nucleotides into duplex DNA in the 5′→3′ direction. Pfu DNA Polymerase also exhibits 3′→5′ exonuclease (proofreading) activity that enables the polymerase to correct nucleotide incorporation errors. It has no 5′→3′ exonuclease activity (see, for example, Thermo Scientific catalog No. EP0501). In some embodiments Klentaq1 is used; it is a Klenow-fragment analog of Taq DNA polymerase, it has no exonuclease or endonuclease activity (see, for example, DNA POLYMERASE TECHNOLOGY, Inc, St. Louis, Mo., catalog No. 100). In some embodiments, the polymerase is a PHUSION DNA polymerase, such as PHUSION High Fidelity DNA polymerase (M0530S, New England BioLabs, Inc.) or PHUSION Hot Start Flex DNA polymerase (M0535S, New England BioLabs, Inc.). In some embodiments, the polymerase is a Q5® DNA Polymerase, such as Q5® High-Fidelity DNA Polymerase (M0491S, New England BioLabs, Inc.) or Q5® Hot Start High-Fidelity DNA Polymerase (M0493S, New England BioLabs, Inc.). In some embodiments, the polymerase is a T4 DNA polymerase (M0203S, New England BioLabs, Inc.).

In some embodiment, between 5 and 600 Units/mL (Units per 1 mL of reaction volume) of polymerase is used, such as between 5 to 100, 100 to 200, 200 to 300, 300 to 400, 400 to 500, or 500 to 600 Units/mL, inclusive.

PCR Methods. In some embodiments, hot-start PCR is used to reduce or prevent polymerization prior to PCR thermocycling. Exemplary hot-start PCR methods include initial inhibition of the DNA polymerase, or physical separation of reaction components reaction until the reaction mixture reaches the higher temperatures. In some embodiments, slow release of magnesium is used. DNA polymerase requires magnesium ions for activity, so the magnesium is chemically separated from the reaction by binding to a chemical compound, and is released into the solution only at high temperature. In some embodiments, non-covalent binding of an inhibitor is used. In this method a peptide, antibody, or aptamer are non-covalently bound to the enzyme at low temperature and inhibit its activity. After incubation at elevated temperature, the inhibitor is released and the reaction starts. In some embodiments, a cold-sensitive Taq polymerase is used, such as a modified DNA polymerase with almost no activity at low temperature. In some embodiments, chemical modification is used. In this method, a molecule is covalently bound to the side chain of an amino acid in the active site of the DNA polymerase. The molecule is released from the enzyme by incubation of the reaction mixture at elevated temperature. Once the molecule is released, the enzyme is activated.

In some embodiments, the amount to template nucleic acids (such as an RNA or DNA sample) is between 20 and 5,000 ng, such as between 20 to 200, 200 to 400, 400 to 600, 600 to 1,000; 1,000 to 1,500; or 2,000 to 3,000 ng, inclusive.

In some embodiments a QIAGEN Multiplex PCR Kit is used (QIAGEN catalog No. 206143). For 100×50 μl multiplex PCR reactions, the kit includes 2× QIAGEN Multiplex PCR Master Mix (providing a final concentration of 3 mM MgCl₂, 3×0.85 ml), 5× Q-Solution (1×2.0 ml), and RNase-Free Water (2×1.7 ml). The QIAGEN Multiplex PCR Master Mix (MM) contains a combination of KCl and (NH₄)₂SO₄ as well as the PCR additive, Factor MP, which increases the local concentration of primers at the template. Factor MP stabilizes specifically bound primers, allowing efficient primer extension by HotStarTaq DNA Polymerase. HotStarTaq DNA Polymerase is a modified form of Taq DNA polymerase and has no polymerase activity at ambient temperatures. In some embodiments, HotStarTaq DNA Polymerase is activated by a 15-minute incubation at 95° C. which can be incorporated into any existing thermal-cycler program.

In some embodiments, 1× QIAGEN MM final concentration (the recommended concentration), 7.5 nM of each primer in the library, 50 mM TMAC, and 7 ul DNA template in a 20 ul final volume is used. In some embodiments, the PCR thermocycling conditions include 95° C. for 10 minutes (hot start); 20 cycles of 96° C. for 30 seconds; 65° C. for 15 minutes; and 72° C. for 30 seconds; followed by 72° C. for 2 minutes (final extension); and then a 4° C. hold.

In some embodiments, 2× QIAGEN MM final concentration (twice the recommended concentration), 2 nM of each primer in the library, 70 mM TMAC, and 7 ul DNA template in a 20 ul total volume is used. In some embodiments, up to 4 mM EDTA is also included. In some embodiments, the PCR thermocycling conditions include 95° C. for 10 minutes (hot start); 25 cycles of 96° C. for 30 seconds; 65° C. for 20, 25, 30, 45, 60, 120, or 180 minutes; and optionally 72° C. for 30 seconds); followed by 72° C. for 2 minutes (final extension); and then a 4° C. hold.

Another exemplary set of conditions includes a semi-nested PCR approach. The first PCR reaction uses 20 ul a reaction volume with 2× QIAGEN MM final concentration, 1.875 nM of each primer in the library (outer forward and reverse primers), and DNA template. Thermocycling parameters include 95° C. for 10 minutes; 25 cycles of 96° C. for 30 seconds, 65° C. for 1 minute, 58° C. for 6 minutes, 60° C. for 8 minutes, 65° C. for 4 minutes, and 72° C. for 30 seconds; and then 72° C. for 2 minutes, and then a 4° C. hold. Next, 2 ul of the resulting product, diluted 1:200, is used as input in a second PCR reaction. This reaction uses a 10 ul reaction volume with 1× QIAGEN MM final concentration, 20 nM of each inner forward primer, and 1 uM of reverse primer tag. Thermocycling parameters include 95° C. for 10 minutes; 15 cycles of 95° C. for 30 seconds, 65° C. for 1 minute, 60° C. for 5 minutes, 65° C. for 5 minutes, and 72° C. for 30 seconds; and then 72° C. for 2 minutes, and then a 4° C. hold. The annealing temperature can optionally be higher than the melting temperatures of some or all of the primers, as discussed herein (see U.S. patent application Ser. No. 14/918,544, filed Oct. 20, 2015, which is herein incorporated by reference in its entirety).

The melting temperature (Tₘ) is the temperature at which one-half (50%) of a DNA duplex of an oligonucleotide (such as a primer) and its perfect complement dissociates and becomes single strand DNA. The annealing temperature (T_{A}) is the temperature one runs the PCR protocol at. For prior methods, it is usually 5° C. below the lowest Tₘ of the primers used, thus close to all possible duplexes are formed (such that essentially all the primer molecules bind the template nucleic acid). While this is highly efficient, at lower temperatures there are more unspecific reactions bound to occur. One consequence of having too low a T_{A} is that primers may anneal to sequences other than the true target, as internal single-base mismatches or partial annealing may be tolerated. In some embodiments of the present inventions, the T_{A} is higher than Tₘ, where at a given moment only a small fraction of the targets have a primer annealed (such as only ˜1-5%). If these get extended, they are removed from the equilibrium of annealing and dissociating primers and target (as extension increases Tₘ quickly to above 70° C.), and a new ˜1-5% of targets has primers. Thus, by giving the reaction a long time for annealing, one can get ˜100% of the targets copied per cycle.

In various embodiments, the annealing temperature is between 1, 2, 3, 4, 5, 6, 7, 8, 9, 10, 11, 12, 13° C. and 2, 3, 4, 5, 6, 7, 8, 9, 10, 11, 12, 13, or 15° C. on the high end of the range, greater than the melting temperature (such as the empirically measured or calculated Tₘ) of at least 25, 50, 60, 70, 75, 80, 90, 95, or 100% of the non-identical primers. In various embodiments, the annealing temperature is between 1 and 15° C. (such as between 1 to 10, 1 to 5, 1 to 3, 3 to 5, 5 to 10, 5 to 8, 8 to 10, 10 to 12, or 12 to 15° C., inclusive) greater than the melting temperature (such as the empirically measured or calculated Tₘ) of at least 25; 50; 75; 100; 300; 500; 750; 1,000; 2,000; 5,000; 7,500; 10,000; 15,000; 19,000; 20,000; 25,000; 27,000; 28,000; 30,000; 40,000; 50,000; 75,000; 100,000; or all of the non-identical primers. In various embodiments, the annealing temperature is between 1 and 15° C. (such as between 1 to 10, 1 to 5, 1 to 3, 3 to 5, 3 to 8, 5 to 10, 5 to 8, 8 to 10, 10 to 12, or 12 to 15° C., inclusive) greater than the melting temperature (such as the empirically measured or calculated Tₘ) of at least 25%, 50%, 60%, 70%, 75%, 80%, 90%, 95%, or all of the non-identical primers, and the length of the annealing step (per PCR cycle) is between 5 and 180 minutes, such as 15 and 120 minutes, 15 and 60 minutes, 15 and 45 minutes, or 20 and 60 minutes, inclusive.

Exemplary Multiplex PCR. In various embodiments, long annealing times (as discussed herein and exemplified in Example 12) and/or low primer concentrations are used. In fact, in certain embodiments, limiting primer concentrations and/or conditions are used. In various embodiments, the length of the annealing step is between 15, 20, 25, 30, 35, 40, 45, or 60 minutes on the low end of the range and 20, 25, 30, 35, 40, 45, 60, 120, or 180 minutes on the high end of the range. In various embodiments, the length of the annealing step (per PCR cycle) is between 30 and 180 minutes. For example, the annealing step can be between 30 and 60 minutes and the concentration of each primer can be less than 20, 15, 10, or 5 nM. In other embodiments the primer concentration is 1, 2, 3, 4, 5, 6, 7, 8, 9, 10, 15, 20, or 25 nM on the low end of the range, and 2, 3, 4, 5, 6, 7, 8, 9, 10, 15, 20, 25, and 50 on the high end of the range.

At high level of multiplexing, the solution may become viscous due to the large amount of primers in solution. If the solution is too viscous, one can reduce the primer concentration to an amount that is still sufficient for the primers to bind the template DNA. In various embodiments, between 1,000 and 100,000 different primers are used and the concentration of each primer is less than 20 nM, such as less than 10 nM or between 1 and 10 nM, inclusive.

Generally speaking, with regard to transplants, the immune system can recognize an allograft as foreign to a body and activate various immune mechanisms to reject the allograft, and it is often necessary to medically suppress the normal immune system response to reject a transplant. Therefore, there is a need for a non-invasive test for transplantation rejection that is more sensitive and more specific than conventional tests. The methods and systems described herein can be used to address this need.

For example, in some embodiments, the present disclosure provides a method for training a neural network using augmented data, including determining, for a training sample, genetic sequencing data or genetic array data for a plurality of genetic positions, determining respective true transplantation rejection state values for a plurality of genetic positions, based on the genetic sequencing data or genetic array data, and determining a neural network comprising one or more layers for calling respective transplantation rejection state values, the neural network defined at least in part by a plurality of weights. The method may further include iteratively modifying the neural network until an exit condition is satisfied, the modifying including determining a batch of data comprising a plurality of cases, each case corresponding to a plurality of genetic positions and comprising data indicating an allele frequency for one or more positions of the respective genetic positions, generating a synthetic case based on one or more of the plurality of cases of the batch, and including the synthetic case in the batch to generate an augmented batch, augmenting the true transplantation rejection state values based on the synthetic case, propagating the batch of data through the neural network to generate a network output comprising one or more respective transplantation rejection state values for each case, and modifying one or more of the plurality of weights based on the network output.

Some embodiments disclosed herein provide for a method of determining the likelihood of transplant rejection within a transplant recipient, the method comprising: a) extracting DNA from the blood sample of the transplant recipient, b) enriching the extracted DNA at target loci, c) amplifying the target loci, and d) measuring an amount of transplant DNA and an amount of recipient DNA in the recipient blood sample, wherein a greater amount of dd-cfDNA indicates a greater likelihood of transplant rejection. Certain neural networks described herein can be used to classify a transplant as being likely to be rejected or unlikely to be rejected, or to classify the likelihood at some greater degree of granularity. For example, a transplant state rejection value can include an amount of dd-cfDNA, an amount of transplant DNA, an amount of recipient DNA, and/or a rejection or success of a transplant. A synthetic case in this regard may include a generated data set (e.g., specifying amount of dd-cfDNA) representing a case for which a “true” value of a transplant state rejection value is that the transplant was rejected. Using techniques described herein, a neural network can be trained to determine a likelihood of success of a transplant, and the neural network can be used to determine or call predict the likelihood of success.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed in connection with one implementation are not intended to be excluded from a similar role in other implementations or implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation, and references to “an implementation,” “some implementations,” “one implementation,” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

As used herein and not otherwise defined, the terms “substantially,” “substantial,” “approximately” and “about”, as well as the symbol “˜” applied to a number (e.g. “˜100”), are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

The indefinite articles “a” and “an,” as used herein in the specification and in the claims, unless clearly indicated to the contrary, should be understood to mean “at least one.”

References to “or” may be construed as inclusive so that any terms described using “or” may indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description, or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

What is claimed is:

1. A method for detecting ploidy state of a fetal chromosome, comprising:
isolating cell-free DNA from a biological sample of a pregnant women comprising a mixture of fetal-derived cell-free DNA and maternal-derived cell-free DNA;
amplifying from the isolated cell-free DNA a plurality of single-nucleotide variant (SNV) loci;
sequencing the amplification products to determine genetic sequencing data or genetic array data of the plurality of SNV loci; and
calling a ploidy state of the fetal chromosome by propagating the sequencing data or genetic array data of the plurality of SNV loci through a neural network.

2. A method for early detection of cancer, comprising:
isolating cell-free DNA from a biological sample of a subject suspected of having cancer comprising a mixture of tumor-derived cell-free DNA and normal tissue-derived cell-free DNA;
amplifying from the isolated cell-free DNA a plurality of single-nucleotide variant (SNV) loci;
sequencing the amplification products to determine genetic sequencing data or genetic array data of the plurality of SNV loci; and
calling a cancer state of the subject by propagating the sequencing data or genetic array data of the plurality of SNV loci through a neural network.

3. A method for detecting cancer relapse or metastasis, comprising:
isolating cell-free DNA from a biological sample of a cancer patient comprising a mixture of tumor-derived cell-free DNA and normal tissue-derived cell-free DNA;
amplifying from the isolated cell-free DNA a plurality of single-nucleotide variant (SNV) loci;
sequencing the amplification products to determine genetic sequencing data or genetic array data of the plurality of SNV loci; and
calling a cancer state of the subject by propagating the sequencing data or genetic array data of the plurality of SNV loci through a neural network.

4. A method for detecting transplantation rejection, comprising:
isolating cell-free DNA from a biological sample of a transplantation recipient comprising a mixture of donor-derived cell-free DNA and recipient-derived cell-free DNA;
amplifying from the isolated cell-free DNA a plurality of single-nucleotide variant (SNV) loci;
sequencing the amplification products to determine genetic sequencing data or genetic array data of the plurality of SNV loci; and
calling a transplantation rejection state of the transplantation recipient by propagating the sequencing data or genetic array data of the plurality of SNV loci through a neural.

5. The method of any of claims 1-4, wherein the neural network comprises one or more layers for calling respective state values, and the neural network is defined at least in part by a plurality of weights.

6. The method of any of claims 1-4, wherein the neural network is obtained by:
determining, for a training sample, genetic sequencing data or genetic array data for a plurality of genetic positions;
determining respective true state values for a plurality of genetic segments, each genetic segment respectively comprising at least some of the plurality of genetic positions, based on the genetic sequencing data or genetic array data;
determining a neural network comprising one or more layers for calling respective state values, the neural network defined at least in part by a plurality of weights;
iteratively modifying the neural network until an exit condition is satisfied, the modifying comprising:
determining a batch of data comprising a plurality of cases, each case corresponding to a respective genetic segment of the plurality of genetic segments and comprising data indicating an allele frequency for one or more positions of the respective genetic segment;
generating a synthetic case based on one or more of the plurality of cases of the batch, and including the synthetic case in the batch to generate an augmented batch;
augmenting the true state values based on the synthetic case;
propagating the batch of data through the neural network to generate a network output comprising one or more respective state values for each case; and
modifying one or more of the plurality of weights based on the network output.

7. The method of any of claims 1-4, wherein the plurality of SNV loci comprises at least 10, or at least 20, or at least 50, or at least 100, or at least 200, or at least 500, or at least 1,000, or at least 2,000, or at least 5,000, or at least 10,000 SNV loci.

8. The method of any of claims 1-4, wherein the amplification products are sequenced with a depth of read of at least 200, or at least 500, or at least 1,000, or at least 2,000, or at least 5,000, or at least 10,000, or at least 20,000, or at least 50,000, or at least 100,000.

9. A method of conducting pre-natal testing, comprising:
determining, for a training sample, genetic sequencing data or genetic array data for a plurality of genetic positions;
determining respective true ploidy state values for a plurality of genetic segments, each genetic segment respectively comprising at least some of the plurality of genetic positions, based on the genetic sequencing data or genetic array data;
determining a neural network comprising one or more layers for calling respective ploidy state values, the neural network defined at least in part by a plurality of weights;
iteratively modifying the neural network until an exit condition is satisfied, the modifying comprising:
determining a batch of data comprising a plurality of cases, each case corresponding to a respective genetic segment of the plurality of genetic segments and comprising data indicating an allele frequency for one or more positions of the respective genetic segment;
generating a synthetic case based on one or more of the plurality of cases of the batch, and including the synthetic case in the batch to generate an augmented batch;
augmenting the true state values based on the synthetic case;
propagating the batch of data through the neural network to generate a network output comprising one or more respective state values for each case; and
modifying one or more of the plurality of weights based on the loss values; and
selecting a test sample comprising plasma extracted from a pregnant mother; and
calling, for the test sample, a ploidy state for a target genetic region by propagating genetic sequencing data for the test sample or genetic array data for the test sample through the modified neural network.

10. The method of claim 9, wherein:
the training sample comprises a plasma sample represented using genetic sequencing data.

11. The method of claim 9, wherein the synthetic case includes a segment that is a homolog of a segment of the one or more of the plurality of cases, and further comprising generating the homolog using a second neural network.

12. The method of claim 11, wherein the second neural network is a generative adversarial network.

13. The method of claim 12, wherein the generative adversarial network includes a generative network trained to generate unphased genotpyes, the method further comprising:
using the unphased genotypes to generate statistics; and
using the statistics to generate the synthetic case.

14. The method of claim 9, wherein the second network includes an autoencoder network.

15. The method of claim 9, wherein generating the synthetic case comprises simulating a chromosomal microdeletion for one of the cases of the plurality of cases.

16. The method of claim 9, wherein:
the test sample comprises a plasma sample, the plasma sample is a mixture of cell-free DNA (cfDNA) from a fetus and host DNA, and the neural networks weights are modified to cause the neural network to better determine the ploidy state of the genetic material from the fetus for a genetic region corresponding to the chromosomal microdeletion.

17. The method of claim 16, wherein the host is a pregnant mother and the plasma sample is a plasma sample of at least the pregnant mother, further comprising using the neural network to predict the occurrence of a specific microdeletion in the fetus of the pregnant mother by passing sequencing data of the pregnant mother's plasma sample through the neural network.

18. The method of claim 17, further comprising generating a plurality of synthetic cases, including the synthetic case, by simulating a chromosomal microdeletion for a plurality of the cases included in the batch, the chromosomal microdeletion being for a specified genetic region.

19. A method of conducting pre-implantation genetic screening, comprising:
determining, for a training sample, genetic sequencing data or genetic array data for a plurality of genetic positions;
determining respective true ploidy state values for a plurality of genetic segments, each genetic segment respectively comprising at least some of the plurality of genetic positions, based on the genetic sequencing data or genetic array data;
determining a neural network comprising one or more layers for calling respective ploidy state values, the neural network defined at least in part by a plurality of weights;
iteratively modifying the neural network until an exit condition is satisfied, the modifying comprising:
determining a batch of data comprising a plurality of cases, each case corresponding to a respective genetic segment of the plurality of genetic segments and comprising data indicating an allele frequency for one or more positions of the respective genetic segment;
generating a synthetic case based on one or more of the plurality of cases of the batch, and including the synthetic case in the batch to generate an augmented batch;
augmenting the true state values based on the synthetic case;
propagating the batch of data through the neural network to generate a network output comprising one or more respective state values for each case; and
modifying one or more of the plurality of weights based on the loss values; and
selecting a test sample from an embryo; and
calling, for the test sample, a ploidy state for a target genetic region by propagating genetic sequencing data for the test sample or genetic array data for the test sample through the modified neural network.

20. The method of claim 19, wherein:
the test sample comprises the embryonic sample and at least one of a maternal sample and a paternal sample, and specifies at least one of a maternal allele frequency and a paternal allele frequency.

21. The method of claim 19, wherein the modifying further comprises perturbing the batch of data prior to propagating the batch of data through the neural network.

22. The method of claim 21, wherein perturbing the batch of data comprises permuting a plurality of array reads for single nucleotide polymorphisms by multiplying the array reads by respective scalars.

23. The method of claim 19, wherein the exit condition is based on at least some of the one or more loss values being equal to or below a predetermined threshold.

24. The method of claim 19, wherein determining, for the training sample, genetic sequencing data or genetic array data for a plurality of genetic positions comprises:
isolating cell-free DNA from a biological sample of a subject;
amplifying from the isolated cell-free DNA a plurality of single-nucleotide variant (SNV) loci that comprise a plurality of target bases; and
sequencing the amplification products to obtain sequence reads of one or more of the plurality of target bases.

25. The method of claim 24, wherein the plurality of target bases comprises at least 10, or at least 20, or at least 50, or at least 100, or at least 200, or at least 500, or at least 1,000 SNV loci.

26. The method of claim 24, wherein the amplification products are sequenced with a depth of read of at least 200, or at least 500, or at least 1,000, or at least 2,000, or at least 5,000, or at least 10,000, or at least 20,000, or at least 50,000, or at least 100,000.

27. A method of training a neural network using augmented data, comprising:
determining, for a training sample, genetic sequencing data or genetic array data for a plurality of genetic positions;
determining respective true state values for a plurality of genetic segments, each genetic segment respectively comprising at least some of the plurality of genetic positions, based on the genetic sequencing data or genetic array data;
determining a neural network comprising one or more layers for calling respective state values, the neural network defined at least in part by a plurality of weights;
iteratively modifying the neural network until an exit condition is satisfied, the modifying comprising:
determining a batch of data comprising a plurality of cases, each case corresponding to a respective genetic segment of the plurality of genetic segments and comprising data indicating an allele frequency for one or more positions of the respective genetic segment;
generating a synthetic case based on one or more of the plurality of cases of the batch, and including the synthetic case in the batch to generate an augmented batch;
augmenting the true state values based on the synthetic case;
propagating the batch of data through the neural network to generate a network output comprising one or more respective state values for each case; and
modifying one or more of the plurality of weights based on the network output.

28. The method of claim 27, wherein generating the synthetic case comprises:
selecting a portion of a first segment of a first case of the plurality of cases;
selecting a portion of a second segment of a second case of the plurality of cases; and
replacing the portion of the first segment with the portion of the second segment.

29. The method of claim 28, further comprising determining the second segment has an aneuploidy based on the true state values, wherein selecting the portion of the second segment is based on the determination that the second segment has an aneuploidy.

30. The method of claim 27, wherein the genetic sequencing data or genetic array data comprises a Cyto12b array or a targeted single nucleotide polymorphism (SNP) pool.

31. The method of claim 27, wherein the genetic sequencing data comprises a number of read counts.

32. The method of claim 27, wherein:
the plasma sample represents a mixture of genetic data targeting germline and somatic variants from a host, and the neural network weights are modified to better quantify the amount of cancerous somatic variants in the plasma.

33. The method of claim 32, further comprising using the neural network to predict the occurrence of cancer in at least one human host.

34. A system for training a neural network for calling a subchromosomal ploidy state, comprising:
a processor; and
processor-executable instructions stored on non-transitory memory that, when executed by the processor, cause the processor to:
determine, for a training sample, genetic sequencing data or genetic array data for a plurality of genetic positions;
determine respective true state values for a plurality of genetic segments, each genetic segment respectively comprising at least some of the plurality of genetic positions, based on the genetic sequencing data or genetic array data;
determine a neural network comprising one or more layers for calling respective state values, the neural network defined at least in part by a plurality of weights;
iteratively modify the neural network until an exit condition is satisfied, the modifying comprising:
determining a batch of data comprising a plurality of cases, each case corresponding to a respective genetic segment of the plurality of genetic segments and comprising data indicating an allele frequency for one or more positions of the respective genetic segment;
selecting a portion of a first segment of a first case of the plurality of cases;
selecting a second segment of a second case of the plurality of cases that has an aneuploidy based on the true state values;
selecting a portion of the second segment;
replacing the portion of the first segment with the portion of the second segment to generate a synthetic case, and including the synthetic case in the batch to generate an augmented batch;
augmenting the true state values based on the synthetic case;
propagating the batch of data through the neural network to generate a network output comprising one or more respective state values for each case; and
modifying one or more of the plurality of weights based on the network output.

35. The system of claim 34, wherein selecting the portion of the first segment comprises selecting a first continuous portion, and wherein selecting the portion of the second segment comprises selecting a second continuous portion.

36. The system of claim 35, wherein the selecting the portion of the first segment comprises selecting a start location for the first segment using a stochastic process.

37. The system of claim 36, wherein the portion of the second segment is selected to have a same start location as the first segment.

38. A method of calling a ploidy state using a neural network, comprising:
determining, for a training sample, genetic sequencing data or genetic array data for a plurality of genetic positions;
determining respective true ploidy state values for a plurality of genetic segments, each genetic segment respectively comprising at least some of the plurality of genetic positions, based on the genetic sequencing data or genetic array data;
determining a neural network comprising one or more layers for calling respective ploidy state values, the neural network defined at least in part by a plurality of weights;
iteratively modifying the neural network until an exit condition is satisfied, the modifying comprising:
determining a batch of data comprising a plurality of cases, each case corresponding to a respective genetic segment of the plurality of genetic segments and comprising data indicating an allele frequency for one or more positions of the respective genetic segment;
propagating the batch of data through the neural network to generate a network output comprising one or more respective ploidy state values for each case;
determining one or more loss values based on the one or more respective ploidy state values, using a loss function and the true ploidy state values; and
modifying one or more of the plurality of weights based on the loss values; and
calling, for a test sample, a ploidy state for a target genetic region by propagating genetic sequencing data for the test sample or genetic array data for the test sample through the modified neural network.

39. The method of claim 38, wherein:
the plurality of genetic positions is a first number of genetic positions,
the plurality of cases is a second number of cases, and
propagating the batch of data through the neural network comprises propagating a tensor through the neural network, the tensor having a first dimension having a length corresponding to the first number, a second dimension having a length corresponding to the second number, and a third dimension having a length corresponding to a third number of data channels.

40. The method of claim 39, wherein:
the training sample comprises an embryonic sample, a maternal sample, and a paternal sample, and
the data channels comprise at least an embryonic allele frequency, a maternal allele frequency, and a paternal allele frequency.

41. The method of claim 39, wherein:
the training sample comprises a plasma sample, and
the data channels comprise a plasma allele frequency.

42. The method of claim 39, wherein the network output comprises a plurality of sets of results comprising a respective result for each data channel, each set of results being specific to at least a respective genetic position of the plurality of genetic positions.

43. The method of claim 38, wherein the modifying further comprises perturbing the batch of data prior to propagating the batch of data through the neural network.

44. The method of claim 38, wherein the training sample is selected from blood, serum, plasma, urine, and a biopsy sample.

45. The method of claim 38, wherein the plurality of target bases are selected from SNV loci identified in the TCGA and COSMIC data sets.

46. A method of training a neural network using augmented data, comprising:
determining, for a training sample, genetic sequencing data or genetic array data for a plurality of genetic positions;
determining respective true cancer state values for a plurality of genetic positions, based on the genetic sequencing data or genetic array data;
determining a neural network comprising one or more layers for calling respective cancer state values, the neural network defined at least in part by a plurality of weights;
iteratively modifying the neural network until an exit condition is satisfied, the modifying comprising:
determining a batch of data comprising a plurality of cases, each case corresponding to a plurality of genetic positions and comprising data indicating an allele frequency for one or more positions of the respective genetic positions;
generating a synthetic case based on one or more of the plurality of cases of the batch, and including the synthetic case in the batch to generate an augmented batch;
augmenting the true cancer state values based on the synthetic case;
propagating the batch of data through the neural network to generate a network output comprising one or more respective cancer state values for each case; and
modifying one or more of the plurality of weights based on the network output.

47. A method of training a neural network using augmented data, comprising:
determining, for a training sample, genetic sequencing data or genetic array data for a plurality of genetic positions;
determining respective true transplantation rejection state values for a plurality of genetic positions, based on the genetic sequencing data or genetic array data;
determining a neural network comprising one or more layers for calling respective transplantation rejection state values, the neural network defined at least in part by a plurality of weights;
iteratively modifying the neural network until an exit condition is satisfied, the modifying comprising:
determining a batch of data comprising a plurality of cases, each case corresponding to a plurality of genetic positions and comprising data indicating an allele frequency for one or more positions of the respective genetic positions;
generating a synthetic case based on one or more of the plurality of cases of the batch, and including the synthetic case in the batch to generate an augmented batch;
augmenting the true transplantation rejection state values based on the synthetic case;
propagating the batch of data through the neural network to generate a network output comprising one or more respective transplantation rejection state values for each case; and
modifying one or more of the plurality of weights based on the network output.

48. A neural network obtained by the method of claim 27.

49. A neural network obtained by the method of claim 46.

50. A neural network obtained by the method of claim 47.

51. A method for detecting ploidy state of a fetal chromosome, comprising:
isolating cell-free DNA from a biological sample of a pregnant women comprising a mixture of fetal-derived cell-free DNA and maternal-derived cell-free DNA;
amplifying from the isolated cell-free DNA a plurality of single-nucleotide variant (SNV) loci;
sequencing the amplification products to determine genetic sequencing data or genetic array data of the plurality of SNV loci; and
calling a ploidy state of the fetal chromosome by propagating the sequencing data or genetic array data of the plurality of SNV loci through the neural network of claim 48.

52. A method for early detection of cancer, comprising:
isolating cell-free DNA from a biological sample of a subject suspected of having cancer comprising a mixture of tumor-derived cell-free DNA and normal tissue-derived cell-free DNA;
amplifying from the isolated cell-free DNA a plurality of single-nucleotide variant (SNV) loci;
sequencing the amplification products to determine genetic sequencing data or genetic array data of the plurality of SNV loci; and
calling a cancer state of the subject by propagating the sequencing data or genetic array data of the plurality of SNV loci through the neural network of claim 49.

53. A method for detecting cancer relapse or metastasis, comprising:
isolating cell-free DNA from a biological sample of a cancer patient comprising a mixture of tumor-derived cell-free DNA and normal tissue-derived cell-free DNA;
amplifying from the isolated cell-free DNA a plurality of single-nucleotide variant (SNV) loci;
sequencing the amplification products to determine genetic sequencing data or genetic array data of the plurality of SNV loci; and
calling a cancer state of the subject by propagating the sequencing data or genetic array data of the plurality of SNV loci through the neural network of claim 49.

54. A method for detecting transplantation rejection, comprising:
isolating cell-free DNA from a biological sample of a transplantation recipient comprising a mixture of donor-derived cell-free DNA and recipient-derived cell-free DNA;
amplifying from the isolated cell-free DNA a plurality of single-nucleotide variant (SNV) loci;
sequencing the amplification products to determine genetic sequencing data or genetic array data of the plurality of SNV loci; and
calling a transplantation rejection state of the transplantation recipient by propagating the sequencing data or genetic array data of the plurality of SNV loci through the neural network of claim 50.
